(19) United States
(12) Patent Application Publication
(10) Pub. No.: US 2024/0091162 A1
(43) Pub. Date: Mar. 21, 2024

(54) COMPOSITIONS AND METHODS FOR TREATING DEPRESSION AND ANXIETY

(71) Applicant: THE TRUSTEES OF COLUMBIA UNIVERSITY IN THE CITY OF NEW YORK, New York, NY (US)

(72) Inventors: Kara MARGOLIS, New York, NY (US); Mark ANSORGE, New York, NY (US); Kam W. LEONG, New York, NY (US); Letao YANG, New York, NY (US); Yuefei ZHU, New York, NY (US)

(21) Appl. No.: 18/514,903

(22) Filed: Nov. 20, 2023

Related U.S. Application Data
(63) Continuation of application No. PCT/US2022/030224, filed on May 20, 2022.
Application No. PCT/US2022/030224 is a continuation of application No. PCT/US2022/030383, filed on May 20, 2022.
(60) Provisional application No. 63/191,759, filed on May 21, 2021.
(60) Provisional application No. 63/191,586, filed on May 21, 2021.

(51) Int. Cl. A61K 9/50; A61K 9/14; A61K 9/51; A61K 45/06

(52) U.S. Cl. CPC A61K 9/501; A61K 9/143; A61K 9/5115; A61K 45/06

(57) ABSTRACT

The present disclosure provides, inter alia, compositions and methods for treating or ameliorating the effect of a disorder such as, e.g., anxiety or depression in a subject, with less or no off- and/or on-target side effects. Also provided are methods for treating such disorder in a pregnant woman.

### CROSS REFERENCE TO RELATED APPLICATIONS

The present application is a continuation of PCT international application no. PCT/US2022/030224, filed on May 20, 2022, which claims benefit of U.S. Provisional Patent Application Ser. No. 63/191,759, filed on May 21, 2021, U.S. Provisional Patent Application Ser. No. 63/191,586, filed on May 21, 2021, and PCT international application no. PCT/US2022/030383, filed on May 20, 2022, which applications are incorporated by reference herein in their entireties.

### GOVERNMENT FUNDING

This invention was made with government support under grant no. W81XWH-17-1-0166 awarded by the Department of Defense, and grant no. GG017055 awarded by the National Aeronautics and Space Administration (NASA). The government has certain rights in the invention.

### FIELD OF DISCLOSURE

The present disclosure provides, inter alia, compositions and methods for treating or ameliorating the effect of a disorder such as, e.g., anxiety or depression in a subject, with less or no off- and/or on-target side effects. Also provided are methods for treating such disorder in a pregnant woman.

### BACKGROUND OF THE DISCLOSURE

Depression and anxiety are highly common, disabling conditions that have huge detrimental impact on the military and society at large. Depression affects up to 8% of the national population (Brody et al. 2018) and is even more common in the military, with major depression (MD) rates of 13.1% and 12% in military members previously and currently deployed, respectively (Harvard Medical School, 2007; Twenge and Joiner, 2020). Although males in the military have a higher suicide prevalence, MD is also one of the top 3 mental health problems diagnosed in female military affiliates (soldiers, spouses and veterans), with rates as high as 30% (Thibaut, 2017). Alarmingly, MD has been linked to numerous negative outcomes in military personnel including an increased risk of suicide and suicide attempts (Jacobsen et al. 2016). Thus, treatment for anxiety and depression is a critical target for suicide prevention.

Anxiety disorders (AD), like MD, are highly prevalent in the military (>7% in the Army) and are also highly associated with increased suicide risk (Trivedi et al. 2006). AD and MD are also increasing rapidly and are commonly comorbid; A US Census Bureau study showed that, compared to 2019 (pre-COVID-19), U.S. adults in April and May of 2020 (COVID-19 pandemic ongoing) were more than three times as likely to screen positive for depressive disorders, AD or both with more than 1/3 screening positive for both conditions (Gollan et al. 2012). This high prevalence of anxiety and depression in military members and society, and their associated increased suicide risk, magnifies the critical need for adequate treatments for suicide prevention, a position that is also reflected in major reports on suicide prevention, including those from the Department of Defense (DoD) (Sinclair et al. 2009).

Antidepressant medications are a first-line treatment recommendation in the Veterans Administration/DoD Clinical Practice Guidelines for MD (2016). In a care review report of >30,000 U.S. Military Members, 79.2% in the depression cohort received a prescription for an antidepressant (Bassotti, 2000).

Among others, selective serotonin reuptake inhibitors (SSRIs) are a first-line treatment for depression and anxiety and the most widely prescribed medications for both conditions to military members and the general public, in large part due to their strong safety profile, relative to other treatments (Marken and Munro, 2000). Yet, despite their high use, currently available SSRIs, which are all systemically absorbed, have several critical limitations.

Currently available SSRIs (systemically absorbed) are severely limited in efficacy. SSRI treatment results in partial symptom improvement in <50% of depressed individuals and induces remission in only a third (with even lower remission rates in those with concurrent anxiety), indicating that SSRIs do not work in the majority of individuals with MD and even less so in those with concurrent anxiety (Al-Jumah and Qureshi, 2012; Gaynes et al. 2005).

Currently available SSRIs (systemically absorbed) are critically limited by off- and on-target side effects. SSRI intake and compliance are often severely limited by the multiple distressing side effects they incur, including anhedonia and anxiety itself (in up to 65% of patients), which can lead many patients to discontinue the medication (Alwan et al. 2016). SSRIs also cause other adverse side effects that limit their intake, including severe constipation (Maim et al. 2016) and even extrapyramidal symptoms (EPS) (Brody et al. 2018). Factors that lead to non-adherence to antidepressant medications are associated with emotional distress, relapse, higher health care costs, and impaired functioning, all of which are highly associated with increased suicide risk.

Moreover, maternal use of systemically absorbed SSRIs is linked to negative effects on infant brain and gut neurodevelopment and long-term function. Depression is common in pregnant women (14-23%) and many servicewoman or military spouses are of childbearing age, with 40% of active-duty servicewomen under 26 years old (Apter-Levy et al. 2013). Further, pregnant military women are >33% more likely to suffer from mental illness (Apter-Levy et al. 2013). Untreated depression in pregnancy is associated with negative outcomes in children, including an increased risk of attention deficit hyperactivity disorder, anxiety and depression (Alwan et al. 2016), decreased cognitive and social functioning, and consequences on somatic health (O'Connor et al. 2016). It is therefore imperative to treat depression during pregnancy.

SSRIs, the most widely prescribed antidepressants during pregnancy, are used in 6-7% of pregnancies nationwide (Yonkers et al. 2014). The potential effects of SSRIs on the fetus, however, are not benign. All SSRIs readily cross the placenta, entering fetal circulation at 70-80% of maternal serum levels and readily crossing fetal blood-brain and gut epithelial barriers (Andrade et al. 2008). Published key pre-clinical findings have confirmed that developmental SSRI exposure leads to alterations in fetal neurodevelopment, brain wiring, and an increased risk of depression, and gastrointestinal (GI) dysfunction (constipation) that persist long-term (Gentile, 2005 (1); Gentile, 2005 (2); Hendrick et al. 2003). These data have been confirmed in human studies showing that in utero SSRI exposure is associated with a significantly higher risk of mood disorders (e.g., anxiety and depression) and/or constipation (Nijenhuis et al. 2012 (Part 1); Nijenhuis et al. 2012 (Part 2)).

Altogether, these data show that there is a critical need for novel ways to treat anxiety and depression that: (1) increase efficacy; (2) avoid side effects (e.g., anxiety, depression and constipation) and; (3) limit the gestational effects of systemic SSRIs on fetal brain and gut neurodevelopment.

### SUMMARY OF THE DISCLOSURE

The shortcomings of SSRIs have long been recognized and strategies have been implemented to improve efficacy and reduce side effect profiles, with limited success. Prior targeting strategies, to bypass these shortcomings, have included efforts to identify critical serotonin (5-HT) receptors through which beneficial effects are mediated, and to then target those receptors directly with selective agonists. A second strategy seeks to identify 5-HT receptors that mediate the adverse effects and to then target those receptors with selective antagonists in conjunction with SSRI treatment (Brody et al. 2018). The present disclosure provides a novel, unconventional strategy to selectively target only a critical subpopulation (gut epithelial) of 5-HT transporters, thereby (1) maintaining efficacy but (2) reducing side effects and (3) avoiding fetal exposure.

Accordingly, one embodiment of the present disclosure is a method for treating or ameliorating the effect of a disorder in a subject. This method comprises administering to the subject an effective amount of an agent that selectively antagonizes intestinal mucosal serotonin reuptake transporter (SERT) with limited or no passage through the intestinal epithelial barrier.

Another embodiment of the present disclosure is a method for treating or ameliorating the effect of a disorder in a pregnant subject while preventing a negative effect on the fetus. This method comprises administering to the pregnant subject an effective amount of an agent that selectively antagonizes intestinal mucosal serotonin reuptake transporter (SERT) with limited or no passage through the intestinal epithelial barrier.

Still another embodiment of the present disclosure is a composition for treating or ameliorating the effect of a disorder in a subject. This composition comprises a gut epithelial-restricted delivery system comprising a particle-based control release device and an agent disposed on a surface of the device, wherein the agent, upon release from the surface of the device, selectively antagonizes intestinal mucosal serotonin reuptake transporter (SERT) with limited or no passage through the intestinal epithelial barrier.

Yet another embodiment of the present disclosure is a method of treating or ameliorating the effect of a disorder in a subject. This method comprises administering to the subject an effective amount of a composition disclosed herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present disclosure. The disclosure may be better understood by reference to one or more of these drawings in combination with the detailed description of specific embodiments presented herein.

FIG. 1 shows an Open-field test that quantifies anxiety-related behaviors. The SERT^{FL/FL}::Villin-cre^{ERT2} mice, when administered tamoxifen, undergo ablation of SERT-containing intestinal epithelial cells, thus mimicking an acute effect of epithelial-restricted SSRI administration. The SERT^{FL/FL}::Villin-cre^{ERT2} mice status post Tamoxifen administration exhibit an anxiolytic phenotype in the open-field test, compared to WT mice with regards to increased vertical head counts and increased ambulatory distance, both total and in the center, compared to WT mice (n=7/8 mice per group). *, **=p<0.05, <0.01, respectively.

FIG. 2 shows that mice lacking epithelial SERT (Villin-cre::SERT^{FL/FL}) exhibit an anxiolytic phenotype compared to WT mice as demonstrated, in the Open Field test, by increased time in the center of the field and an increased # of head rearings. In contrast, mice lacking SERT in the ENS (Wnt1-cre::SERT^{FL/FL}) exhibit an anxiogenic phenotype in the same parameters. *, **, ***=p<0.05, <0.01, and <0.001, respectively.

FIG. 3 shows that mice lacking epithelial SERT (Villin-cre::SERT^{FL/FL}) exhibit an anxiolytic phenotype compared to WT mice as demonstrated, in the Novelty Suppressed Feeding Test, by decreased time in latency to eat and no difference in food consumption (figs above) or weight change (not shown). In contrast, mice lacking SERT in the ENS (Wnt1-cre::SERT^{FL/FL}) exhibit an anxiogenic phenotype in the same parameter. *=p<0.05.

FIG. 4 shows that mice lacking epithelial SERT (Villin-cre::SERT^{FL/FL}) exhibit significantly less behavioral despair (BD) compared to WT mice, as demonstrated in the Tail Suspension Test, by decreased levels of immobility. In contrast, mice lacking SERT in the ENS (Wnt1-cre::SERT^{FL/FL}) exhibit increased BD as demonstrated, in the Learned Helplessness Test, by an increased time of latency to escape. *=p<0.05.

FIG. 5 shows that mice lacking epithelial SERT (Villin-cre::SERT^{FL/FL}) do not exhibit slowed colonic motility compared to WT mice. In contrast, mice lacking SERT in the ENS (Wnt1-cre::SERT^{FL/FL}) exhibit slowed colonic motility (constipation), when compared to WT mice. **=p<0.01.

FIG. 6 shows acute ablation of SERT in the gut epithelium (after Tamoxifen administration) in Villin-cre-ERT2::SERT^{FL/FL} mice compared to WT as evidenced by increased total ambulatory distance and entries in to the center, in the Open Field Test. *=p<0.05.

FIG. 7 shows that acute ablation of SERT in the gut epithelium (after Tamoxifen administration) in Villin-cre-ERT2:: SERT^{FL/FL} mice compared to WT is anxiolytic as evidenced by significantly more entries into the center in the Open Field Test (males and females combined).

FIG. 8 shows that acute ablation of SERT in the gut epithelium (after Tamoxifen administration) in Villin-cre-ERT2::SERT^{FL/FL} mice compared to WT is anxiolytic as evidenced by significantly decreased latency to touch paper in the novelty-suppressed feeding test (males and females combined).

FIGS. 9A-9F show that SERT deletion from enteric and central neurons or the gut epithelium does not impact cognition. Wnt-1^{Cre}::SERT^{fl/fl} mice do not display altered freezing response in the fear extinction paradigm (FIG. 9A) or any significant changes during the Morris Water Maze test in both the latency to platform during spatial learning task (FIG. 9B) and the number of platform crossings in the probe trial (FIG. 9C). Compared to control mice, Villin^{Cre}::SERT^{fl/fl} mice exhibit no freezing behavior differences during the fear extinction paradigm (FIG. 9D), a similar latency time to platform during training (FIG. 9E) and platform crossings in the probe test (FIG. 9F) of the Morris Water Maze test.

FIG. 10 shows that mice that lack SERT in the ENS (Wnt1-cre::SERT^{FL/FL}; W/S) exhibit increased visceral sensitivity to colorectal distension (CRD; by balloon distention) as measured by visceromotor response (VMR). W/S mice show increased sensitivity to increasing CRD pressures, compared to WT. *=P<0.05.

FIGS. 11A-11C show that SERT deletion from the gut epithelium does not alter serotonergic innervation in the dorsal and ventral hippocampus or the medial prefrontal cortex. Assessment of serotonergic neuron density in different brain regions of mice with specific SERT deletion from the gut epithelium. The left panel shows representative confocal images of coronal sections with staining of SERT-positive fibers in the dorsal (FIG. 11A) and ventral (FIG. 11B) CA1 region of the hippocampus, and in the mPFC (FIG. 11C), Scale bars, 50 μm. The right panel shows quantification of relative optical density of SERT staining in Villin^{Cre}::SERT^{fl/fl} and control mice, n=4-5/group. Data are presented as mean±SEM. There are no differences in the density of SERT-positive serotonergic fibers in different subregions of hippocampal CA1 and mPFC. Two-way ANOVA was used to compare groups in the different subregions. SERT: serotonin transporter, SO: stratum oriens, SP: stratum pyramidale, SR: stratum radiatum, SLM: stratum lacunosum-moleculare, SM: stratum moleculare, dCA1: dorsal cornus ammonis 1, vCA1: dorsal cornus ammonis 1, PrL: prelimbic, IL; infralimbic.

FIGS. 12A-12H show that total neuronal numbers are unchanged in the ENS of Wnt-1^{Cre}::SERT^{fl/fl} and Villin^{Cre}::SERT^{fl/fl} mice but CHAT- and NOS-expressing neurons are increased significantly in Wnt-1^{Cre}::SERT^{fl/fl} mice. Representative immunofluorescence staining of ChAT (FIG. 12A) and NOS (FIG. 12B) neurons (white arrows) in colonic myenteric plexus preparations of WT and Wnt-1^{Cre}::SERT^{fl/fl} colons. Scale bars, 50 μm. Quantification of total neurons (FIG. 12C) and proportions of cholinergic, ChAT neurons (FIG. 12D) and NOS neurons (FIG. 12E) in the myenteric plexus of Wnt-1^{Cre}::SERT^{fl/fl} mice, n=5-10/group. Quantification of total neurons (FIG. 12F) and proportions of ChAT-s (FIG. 12G) and NOS-expressing neurons (FIG. 12H) in the myenteric plexus of WT and Villin^{Cre}::SERT^{fl/fl} mice.

FIGS. 13A-13I show that selective deletion of SERT from the ENS results in hyperplasia of serotonergic varicosities. FIGS. 13A and 13C show the results of 5-HT axonal projections in the MP of the colons of Wnt-1^{Cre}::SERT^{fl/fl} (FIG. 13A) and Villin^{Cre}::SERT^{fl/fl} (FIG. 13C) mice. FIGS. 13C-13E show that 5-HT axonal projections and the proportion of 5-HT neurons in the MP of Villin^{Cre}::SERT^{fl/fl} mice were not significantly different compared to WT. FIG. 13B shows that serotonergic axonal projections were significantly reduced in the MP of Wnt-1^{Cre}::SERT^{fl/fl} mice compared to WT mice while the accumulation of 5-HT into neuronal cell bodies was undetected. FIGS. 13F and 13G show the results of analysis of 5-HT at a high power objective to quantify its distribution within each ganglion, indicating that Wnt-1^{Cre}::SERT^{fl/fl} mice had significantly more 5-HT varicosities within the ganglion compared to WT. FIGS. 13H and 13I show the results of the same analysis as FIGS. 13F and 13G in the Villin^{Cre}::SERT^{fl/fl} m ice.

FIG. 14 shows the effective and selective delivery of SSRIs into intestinal epithelium using a microbur technology platform, including the overall design of the microbur drug delivery device and representative electron microscopy images for microburs and nanoparticles, and schematic diagrams showing how orally administered microburs enhance the retention and selective delivery of SSRIs into intestinal epithelium.

FIGS. 15A-15G show some preliminary data for microbur drug delivery platform-based release of SSRIs.

FIG. 15A shows the overall material design of microburs.

FIG. 15B is a representative scanning electron microscopy image showing the morphology of nanoparticle-loaded microbur.

FIG. 15C shows enhanced mucoadhesion of microburs studied by treating confluent HCT116 cell culture with microburs (5 mg/mL) or control nanoparticles (500 nm polystyrene beads) for 15 minutes followed by quantification on the remaining areas after water flushing. n=4 biological replicates. ***P<0.001.

FIG. 15D is a graph showing the excellent acid (pH=1) stability and mucus-specific (under 100 mM glutathione, or “GSH,”) biodegradation of microburs. Absorbance corresponds to the concentration of microburs.

FIG. 15E shows controlled release of the proposed polymer nanoparticles from the microbur after treatment by 1 mM GSH.

FIG. 15F shows mesoporous silica nanoparticles (MSNs) with tunable sizes from 10 nm to over 200 nm synthesized in the Leong lab, which can be used to modulate SSRI release.

FIG. 15G shows controlled release of a sample SSRI (Fluoxetine) from a 42 nm-sized MSN. Inset photograph shows the fluorescence of dye-doped MSNs for potentially tracking drugs and nanoparticle biodistributions ex vivo and in vivo.

FIGS. 16A and 16B show sustained gut retention of microburs (MBs) on the intestinal epithelium.

FIG. 16C shows the excellent stability of MB in acidic conditions and biodegradability in mucus glutathione (GSH).

FIG. 16D shows modulation of sustainable FLX release profile from MB with different structures.

FIG. 17 shows the results of in vivo FLX pharmacokinetic study illustrating the local dispersion of FLX on small intestine epithelium with minimal systemic biodistribution after 12-hours of FLX release from microburs (MBs) (left) and the in vivo imaging system analysis of model drug (Fluorescein) distribution after delivery by MB, showing the selective dispersion of drug in the gut (right).

FIG. 18 shows the in vivo biosafety of MB delivery system based on: the minimal disruption of tight junction as shown by no difference in intestinal epithelial permeability (left panel, n=3, *P<0.05) and no weight loss after repeated oral gavage of mice with MB-MSN-SSRIs with FLX concentrations>10-fold above standard dosing of FLX (right panel, n=5; dose administered=250 mg/kg).

FIG. 19 shows that SERT^{FL/FL}/villin^{Cre-ERT2} mice exhibit less sensitivity to colorectal distention, as measured by visceromotor response.

### DETAILED DESCRIPTION OF THE DISCLOSURE

Selective serotonin reuptake inhibitors (SSRIs) are a current first-line therapy for many depressive and anxiety-related disorders yet have low efficacy (<50%). All available SSRIs are systemically active. Even when such SSRIs are effective, they are fraught with side effects significant enough to cause their discontinuation (e.g., anxiety, constipation, and even extrapyramidal symptoms (EPS) in rare cases). In the present disclosure, it is found that: (1) chronic SSRI exposure acts on 5-HT2C receptors to inhibit central nervous system (CNS) dopamine activity, which produces motor side effects and diminishes antidepressant and anxiolytic efficacy, showing that SSRI exposure to the CNS can have deleterious effects on mood; (2) blockade of SERT in the enteric nervous system (ENS) causes anxiety and depression-like phenotypes, as well as constipation, showing that SSRI exposure to the ENS can also be harmful; and (3) blockade of intestinal epithelial SERT (without alteration of SERT in the CNS or the ENS) alleviates anxiety- and depression-like phenotypes. These three findings strongly support the notion that a drug delivery system that can disperse fluoxetine (a SSRI tested herein) in a more gut epithelial-restricted manner, and limit systemic absorption (e.g., to ENS and CNS), would be sufficient and potentially even more effective at treating anxiety-and depression-related disorders while avoiding negative side effects.

Accordingly, the present disclosure relates to an intestine mucosa-specific drug that does not enter the blood stream and therefore does not have off target effects. One embodiment of the present disclosure is a method for treating or ameliorating the effect of a disorder in a subject. This method comprises administering to the subject an effective amount of an agent that selectively antagonizes intestinal mucosal serotonin reuptake transporter (SERT) with limited or no passage through the intestinal epithelial barrier.

In some embodiments, the agent has limited or no effect on SERT in central nervous system (CNS) and enteric nervous system (ENS).

In some embodiments, the disorder is selected from the group consisting of: a gastrointestinal disorder, a central nervous system disorder, an anxiety disorder, a mood disorder, a depressive disorder, an autism spectrum disorder, a substance abuse or dependence disorder, an attention deficit hyperactivity disorder (ADHD), a post-traumatic stress disorder (PTSD), and combinations thereof.

In some embodiments, the gastrointestinal disorder is selected from the group consisting of: abdominal pain, constipation, nausea, intestinal inflammatory disease, disorders of gut-brain interactions (e.g., irritable bowel syndrome), enteric nervous system hyperplasia, Crohn's disease, ulcerative colitis, microscopic colitis, and combinations thereof. In some embodiments, the gastrointestinal disorder is abdominal pain.

In some embodiments, the agent is selected from the group consisting of: a selective serotonin reuptake inhibitor (SSRI), a serotonin-norepinephrine reuptake inhibitor (SNRI), a tricyclic antidepressant (TCA), an atypical antidepressant, and combinations thereof. In some embodiments, the agent is an SSRI.

In some embodiments, the selective antagonism of intestinal mucosal SERT is achieved with assistance of a gut epithelial-restricted delivery system. In some embodiments, the gut epithelial-restricted delivery system is a bio-microbur therapeutic delivery platform comprising: a spherical, hollow core having a surface, and a plurality of nanoneedles secured to the surface of the core and extending outwardly therefrom. The density of the nanoneedles on the surface can be in the range of 10 million per square centimeter to 100 per square centimeter. The vertically aligned nanoneedles enhance the GI retention of the device by increasing their surface areas, providing anchoring points to the mucus layer, thereby enhancing their adhesion to the mucosal layer. Therapeutic agents such as SSRIs will be encapsulated or absorbed into the bio-microbur through physical absorption or covalent conjugation. The agents will then be released through passive diffusion or mucus-triggered degradation of the microscale devices and, in a SR-fashion, can be limited in dispersion over time, providing more exposure to the epithelium with minimized systemic absorption.

In some embodiments, the core and nanoneedles comprise manganese oxide (MnO₂). Manganese is an essential element for humans and is commonly seen in daily supplements. Manganese dioxide is stable in acid but biodegradable by stimuli existent in the mucosal layer. This would allow manganese dioxide bio-microbur to selectively release therapeutic agents (e.g., a SSRI) into the mucosal layer. In some embodiments, the core and nanoneedles comprise titanium oxide (TiO₂).

In some embodiments, the core is loaded inside with SSRI encapsulated mesoporous silica nanoparticles. When assembling the delivery system, the selected SSRIs are first loaded into the nanoparticles, and the nanoparticles are then loaded into the hollow core of the bio-microbur platform. The nanoparticles may have sizes ranging from 1-1000 nm and shapes ranging from dots, rods, wires, sheets, and stars. The nanoparticles may also have the compositions of liposomes, exosomes, polylactic-co-glycolic acid, polyvinylpyrrolidone, polyethylene glycol, polymethacrylates, gelatin, chitosan, polyethyleneimine, silica, silicon, gold, iron oxide, and quantum dots. In some embodiments, the nanoparticles are made of chitosan-polyethyleneimine-polyboronic acid (chitosan-PEI-PBA).

In some embodiments, the nanoneedles have an average length of about 1 nm to 100 nm. In some embodiments, the bio-microbur has a size in the range of 1 μm to 5 μm. In some embodiments, the bio-microbur can be specifically targeted to different parts of the GI tract based on pH or biological stimuli.

In some aspects of this and other embodiments of the disclosure, the subject is a mammal. Preferably, the mammal is selected from the group consisting of humans, primates, farm animals, and companion animals such as dogs and cats. More preferably, the mammal is a human. The term does not denote a particular age. Thus, both adult and newborn animals, as well as fetuses, are intended to be covered. In some embodiments of the present disclosure, the subject is a pregnant woman.

SSRIs are also the first-line treatment for pregnant women with depression/anxiety. Systemic SSRIs cross through the placenta and the breastmilk and have been shown to affect fetal neurodevelopment and long-term brain and gut functions in negative ways; these children suffer from an increased risk of mood disorders, cognitive issues, attention deficit hyperactivity disorder (ADHD) as well as functional GI disorders. An epithelial-targeted SERT antagonist would minimize systemic absorption and would thus be a safer alternative to treat depression/anxiety during pregnancy.

Accordingly, another embodiment of the present disclosure is a method for treating or ameliorating the effect of a disorder in a pregnant subject while preventing a negative effect on the fetus. This method comprises administering to the pregnant subject an effective amount of an agent that selectively antagonizes intestinal mucosal serotonin reuptake transporter (SERT) with limited or no passage through the intestinal epithelial barrier.

In some embodiments, the agent has limited or no effect on SERT in central nervous system (CNS) and enteric nervous system (ENS).

In some embodiments, the disorder is selected from the group consisting of: a gastrointestinal disorder, a central nervous system disorder, an anxiety disorder, a mood disorder, a depressive disorder, an autism spectrum disorder, a substance abuse or dependence disorder, an attention deficit hyperactivity disorder (ADHD), a post-traumatic stress disorder (PTSD), and combinations thereof.

In some embodiments, the gastrointestinal disorder is selected from the group consisting of: abdominal pain, constipation, nausea, intestinal inflammatory disease, disorders of gut-brain interactions (e.g., irritable bowel syndrome), enteric nervous system hyperplasia, Crohn's disease, ulcerative colitis, microscopic colitis, and combinations thereof. In some embodiments, the gastrointestinal disorder is abdominal pain.

In some embodiments, the agent is selected from the group consisting of: a selective serotonin reuptake inhibitor (SSRI), a serotonin-norepinephrine reuptake inhibitor (SNRI), a tricyclic antidepressant (TCA), an atypical antidepressant, and combinations thereof. In some embodiments, the agent is an SSRI.

In some embodiments, the negative effect on the fetus is selected from the group consisting of deficit gut and/or brain neurodevelopment/function, attention deficit hyperactivity disorder (ADHD), anxiety, depression, decreased cognitive and social functioning, gastrointestinal (GI) mobility disorder, Autism spectrum disorder (ASD), cardiac disorders, and combinations thereof.

In some embodiments, the selective antagonism of intestinal mucosal SERT is achieved with assistance of a gut epithelial-restricted delivery system as disclosed herein.

Still another embodiment of the present disclosure is a composition for treating or ameliorating the effect of a disorder in a subject. This composition comprises a gut epithelial-restricted delivery system comprising a particle-based control release device and an agent disposed on a surface of the device, wherein the agent, upon release from the surface of the device, selectively antagonizes intestinal mucosal serotonin reuptake transporter (SERT) with limited or no passage through the intestinal epithelial barrier.

In some embodiments, the agent has limited or no effect on SERT in central nervous system (CNS) and enteric nervous system (ENS).

In some embodiments, the disorder is selected from the group consisting of: a gastrointestinal disorder, a central nervous system disorder, an anxiety disorder, a mood disorder, a depressive disorder, an autism spectrum disorder, a substance abuse or dependence disorder, an attention deficit hyperactivity disorder (ADHD), a post-traumatic stress disorder (PTSD), and combinations thereof.

In some embodiments, the gastrointestinal disorder is selected from the group consisting of: abdominal pain, constipation, nausea, intestinal inflammatory disease, disorders of gut-brain interactions (e.g., irritable bowel syndrome), enteric nervous system hyperplasia, Crohn's disease, ulcerative colitis, microscopic colitis, and combinations thereof. In some embodiments, the gastrointestinal disorder is abdominal pain.

In some embodiments, the agent is selected from the group consisting of: a selective serotonin reuptake inhibitor (SSRI), a serotonin-norepinephrine reuptake inhibitor (SNRI), a tricyclic antidepressant (TCA), an atypical antidepressant, and combinations thereof. In some embodiments, the agent is an SSRI.

In some embodiments, the particle-based control release device comprises a bio-microbur therapeutic delivery platform comprising: a spherical, hollow core having a surface, and a plurality of nanoneedles secured to the surface of the core and extending outwardly therefrom. In some embodiments, the core and nanoneedles comprise manganese oxide (MnO₂) or titanium oxide (TiO₂). In some embodiments, the core is loaded inside with SSRI encapsulated mesoporous silica nanoparticles. In some embodiments, the nanoneedles have an average length of about 1 nm to 100 nm. In some embodiments, the bio-microbur has a size in the range of 1 μm to 5 μm. In some embodiments, the bio-microbur can be specifically targeted to different parts of the GI tract based on pH or biological stimuli.

To further enhance the localization to intestinal mucosa, in some embodiments, the bio-microbur disclosed herein can be coated with mucoadhesive polymers, such as chitosan, polyacrylates, hyaluronan, cellulose-derived polymers, alginates, gelatin, and pectin. The coating thickness will be less than the nanoneedle tip size, and the coating can be done by either non-covalent (e.g., electrostatic) or covalent (e.g., amidation, thiol-metal coordination, and esterification) method.

Yet another embodiment of the present disclosure is a method of treating or ameliorating the effect of a disorder in a subject. This method comprises administering to the subject an effective amount of a composition disclosed herein.

As used herein, the terms “treat,” “treating,” “treatment” and grammatical variations thereof mean exposing an individual subject to a protocol, regimen, process or remedy, in which it is desired to obtain a physiologic response or outcome in that subject, e.g., a patient. However, because every treated subject may not respond to a particular treatment protocol, regimen, process or remedy, treating or preventing does not require that the desired physiologic response or outcome be achieved in each and every subject or subject population, e.g., patient population. Accordingly, a given subject or subject population, e.g., patient population, may fail to respond or respond inadequately to treatment.

As used herein, the terms “prevent,” “preventing,” or “prevention,” and grammatical variations thereof refer to the prophylactic treatment of a subject in need thereof.

As used herein, the term “disorder” broadly refers to a syndrome, condition, chronic illness or a particular disease. For example, the disorder may be a gastrointestinal disorder or a behavioral disorder. In the present disclosure, a “gastrointestinal disorder” refers to a disease involving the gastrointestinal tract, namely the esophagus, stomach, small intestine, large intestine and rectum, and the accessory organs of digestion, the liver, gallbladder, and pancreas. Thus, as used herein, “gastrointestinal disorder” includes but is not limited to: abdominal pain, constipation, nausea, intestinal inflammatory disease, irritable bowel syndrome, enteric nervous system hyperplasia, Crohn's disease, ulcerative colitis, microscopic colitis, and combinations thereof. The disorder may also be a disorder of the CNS caused by exposure of the subject to an agent, such as an SSRI, while in utero and/or during breastfeeding. Other non-limiting examples of disorders according to the present disclosure include: an anxiety disorder, a mood disorder, a depressive disorder, an autism spectrum disorder, a substance abuse or dependence disorder, an attention deficit hyperactivity disorder (ADHD), a post-traumatic stress disorder (PTSD), and combinations thereof.

As used herein, the term “antagonist” or “antagonism” means a compound or composition that reduces, interferes with, or inhibits physiological activity. For example, an antagonist of the serotonin reuptake transporter (SERT) prevents the removal of serotonin from the synaptic cleft by the transporter. In the present disclosure, an “agent” means a compound or composition that antagonizes SERT. Non-limiting examples of agents of the disclosure include: selective serotonin reuptake inhibitors (SSRIs), serotonin-norepinephrine reuptake inhibitors (SNRIs), tricyclic antidepressants (TCAs), atypical antidepressants, and combinations thereof. Preferably, the agent is an SSRI, such as, e.g., citalopram (Celexa; Forest Labs), escitalopram (Lexapro; Forest Labs), fluoxetine (Prozac; Eli Lilly), fluvoxamine (Luvox; Abbott Laboratories), fluvoxamine CR (Luvox CR; Jazz Pharmaceuticals), paroxetine (Paxil; GlaxoSmithKline), paroxetine CR (Paxil CR; GlaxoSmithKline), and sertraline (Zoloft; Pfizer).

The terms “administering”, “administration” and variants thereof (particularly “administering” an agent or antagonist) as used herein means introducing an agent, e.g., a SSRI, into the body of a subject, such as a human, in need of such treatment.

In the present disclosure, an “effective amount” or a “therapeutically effective amount” of an agent, such as a SSRI, is an amount that is sufficient to effect beneficial or desired results as described herein when administered to a subject. Effective dosage forms, modes of administration, and dosage amounts may be determined empirically, and making such determinations is within the skill of the art. It is understood by those skilled in the art that the dosage amount will vary with the route of administration, the rate of excretion, the duration of the treatment, the identity of any other drugs being administered, the age, size, and species of mammal, e.g., human patient, and like factors well known in the arts of medicine and veterinary medicine. In general, a suitable dose of any active agent disclosed herein will be that amount of the active agent which is the lowest dose effective to produce the desired effect.

The following examples are provided to further illustrate certain aspects of the present disclosure. These examples are illustrative only and are not intended to limit the scope of the disclosure in any way.

### EXAMPLES

### Example 1

### Exclusive Blockade of Intestinal Mucosal SERT

A major obstacle to improving upon currently available (systemic) SSRIs is a lack of understanding of precisely where SSRIs act to induce their anti-depressive and anxiolytic effects. Our extensive data show that selective antagonism of SERT in the gut epithelium is necessary and sufficient to ameliorate anxiety and depression-related behavior. This unexpected breakthrough finding indicates that targeting SSRIs to the gut epithelium may provide a novel way to treat anxiety and depression more effectively.

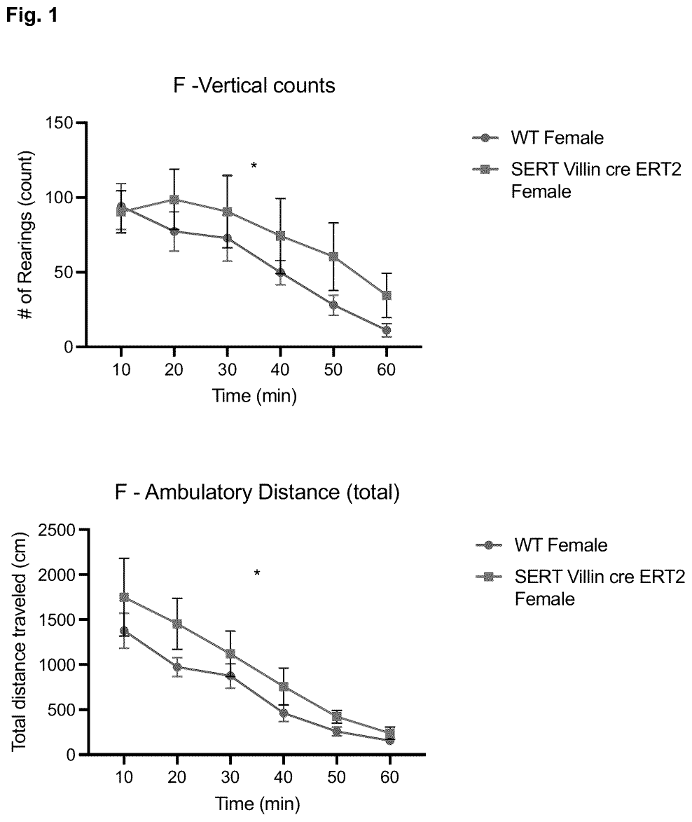

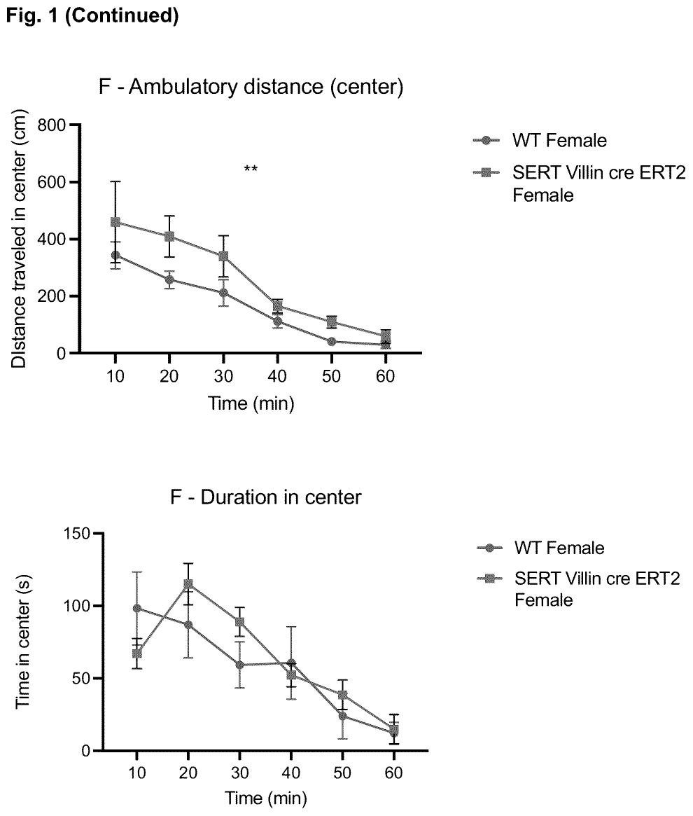

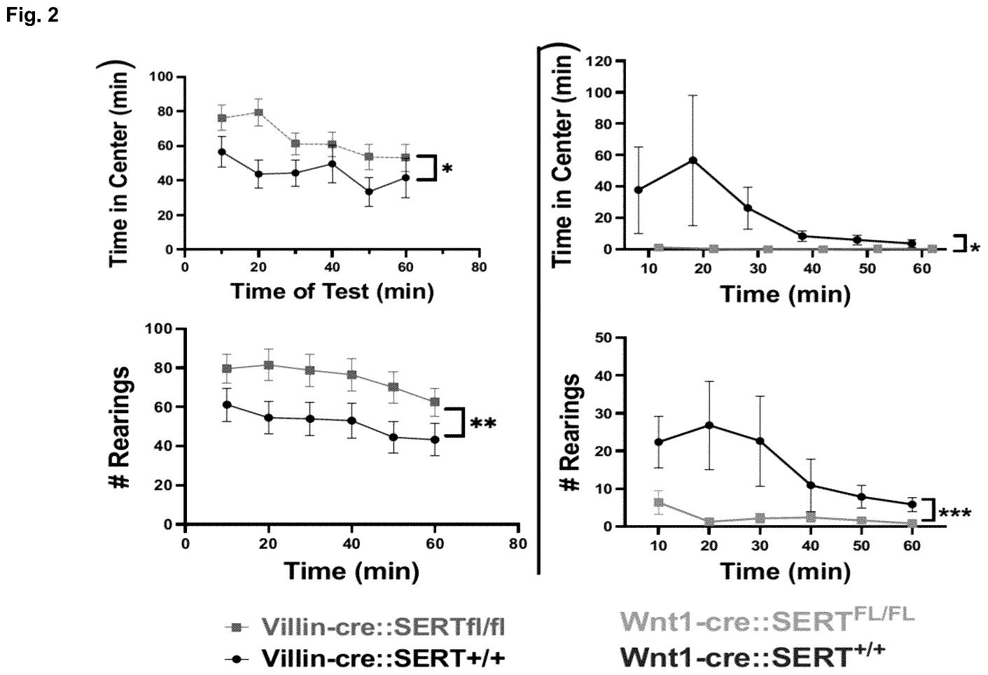

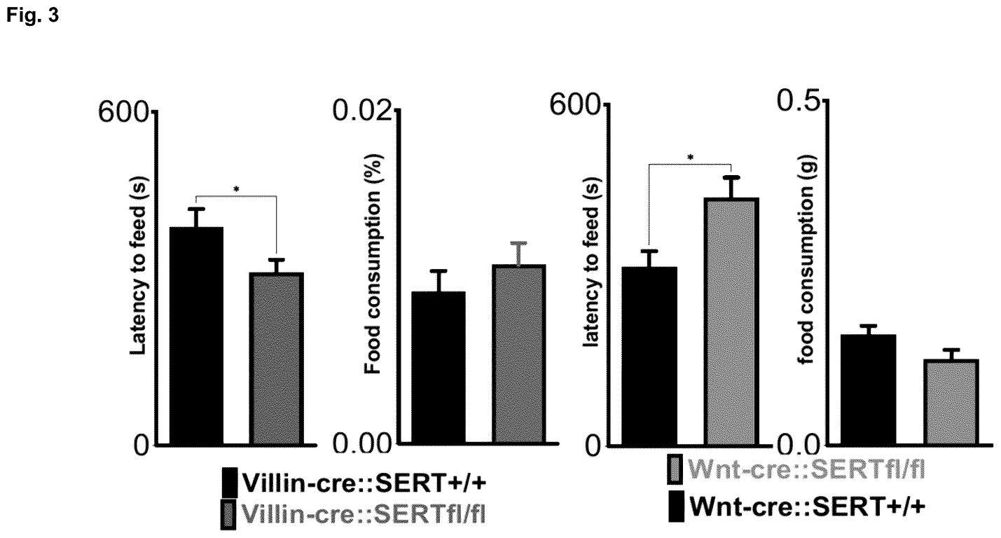

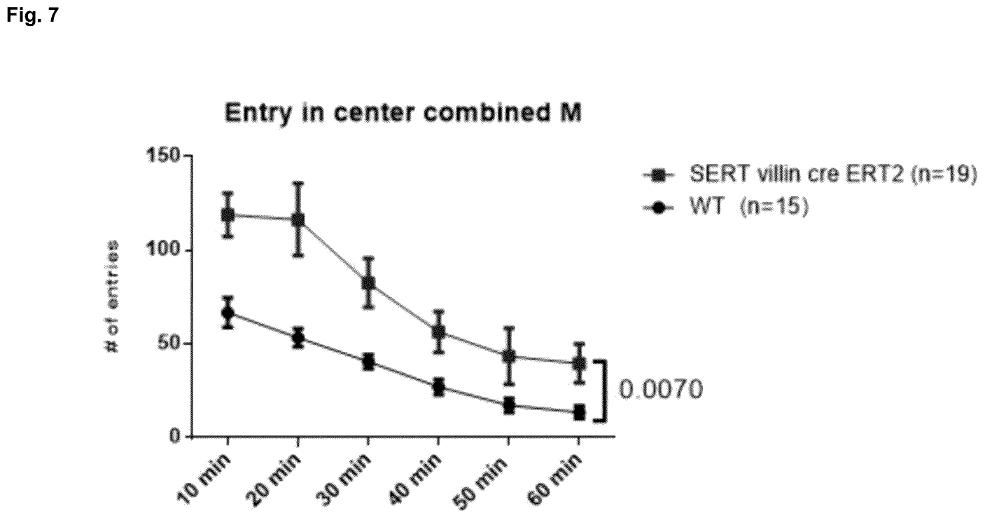

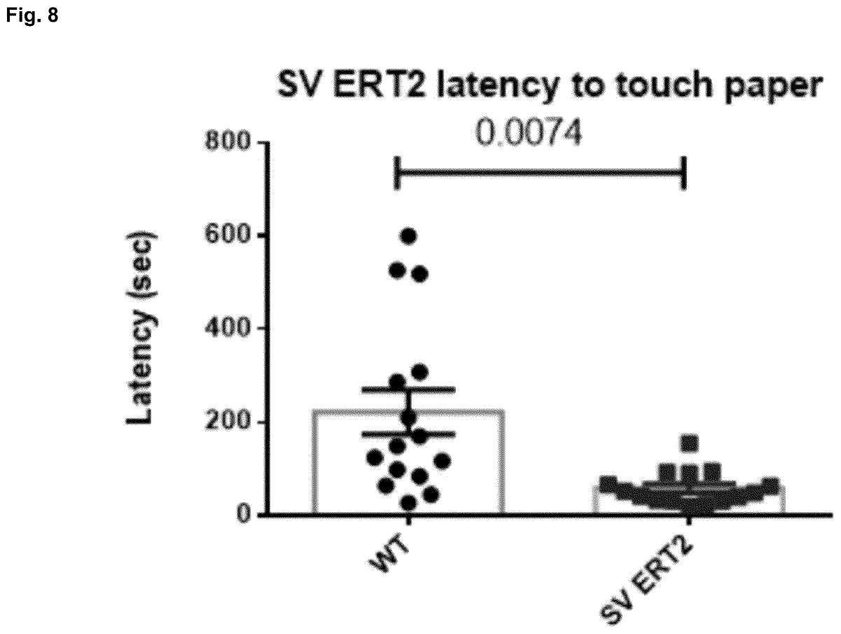

SSRIs function by inhibiting SERT, a transporter critical for 5-HT inactivation, thus increasing serotonergic neurotransmission (Ansorge et al. 2008). SERT is located in the CNS, the gut epithelium and the ENS. SSRIs thus increase serotonergic signaling in all three areas. It has been thought that SSRIs implement their anti-depressive and anxiolytic actions by acting directly on the CNS. It is found that modulation of intestinal 5-HT in the mucosa or the ENS has effects, not only on intestinal motility but also on anxiety and depression (Ansorge et al. 2004; Margolis et al. 2016). To mimic targeted SSRI exposure, we created and studied novel conditional knock out (KO) mice, where SERT was selectively eliminated from the gut epithelium or the ENS. In those mice we examined GI function, anxiety, depression and cognition. We had hypothesized that gut-selective (ENS or gut epithelial) elimination of SERT would affect GI function but not behavior. Indeed, these mice did not display any phenotypes in learning and memory-related behavioral tasks (data not shown). However, to our surprise, ENS and intestinal mucosal SERT inhibition critically impacted emotional behavior (FIGS. 1-13I).

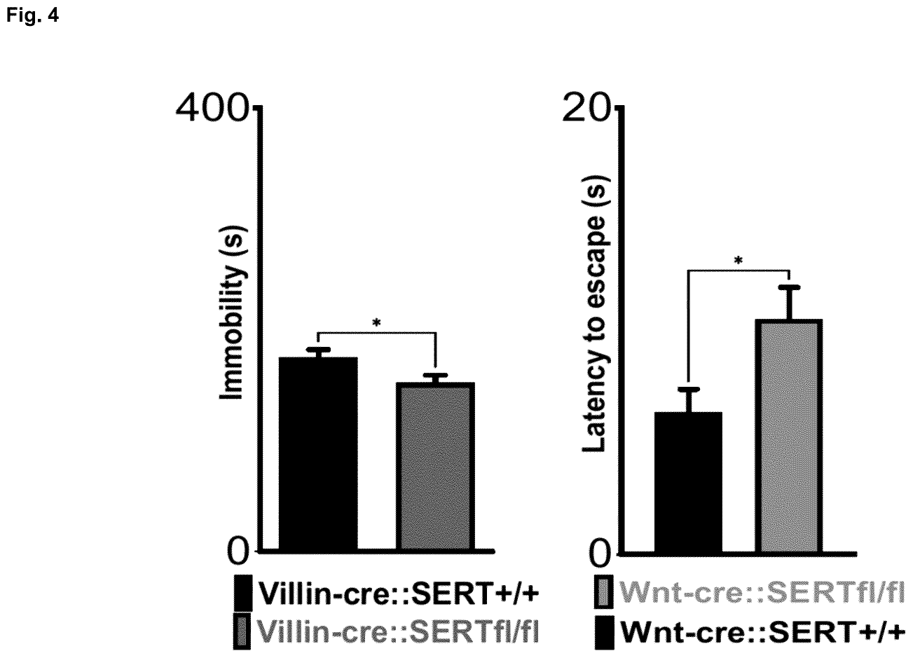

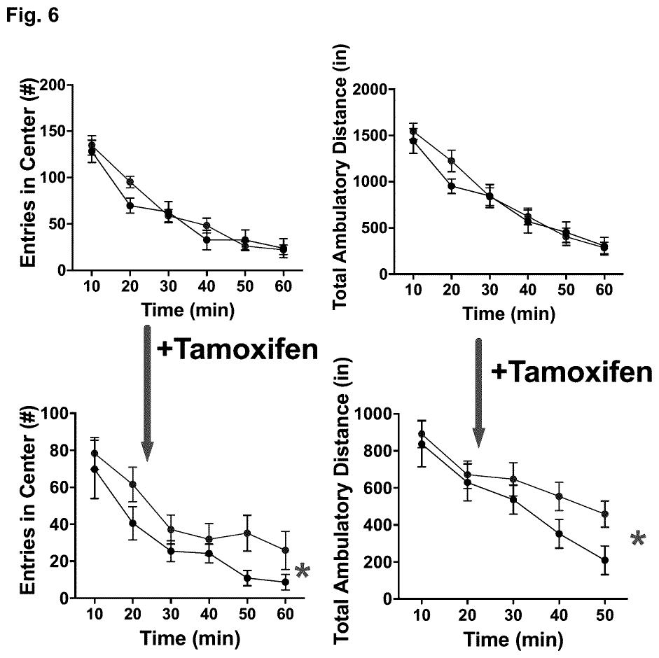

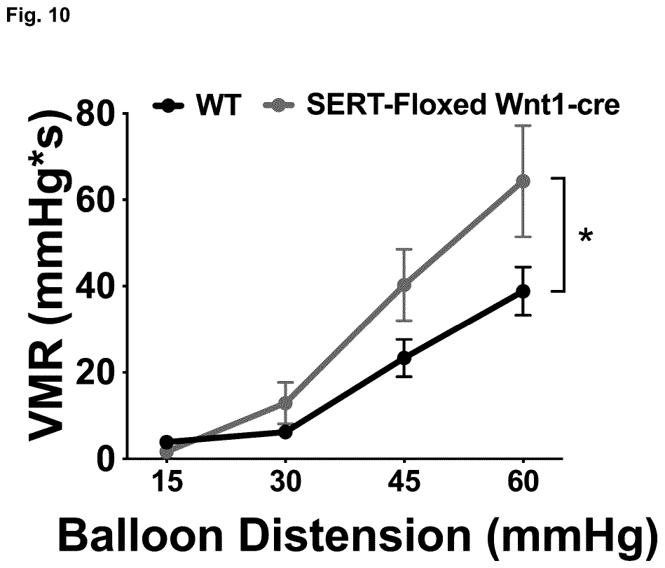

Specifically, mice that had selective deletion of SERT in the intestinal mucosa, without SERT deletion in the CNS or the ENS (villin^{cre} mice:: SERT^{FL/FL}) surprisingly displayed anxiolytic-like phenotypes in the Open Field (OF) and Novelty Suppressed Feeding (NSF) Tests (FIGS. 1-3) and significantly less behavioral despair in the Tail Suspension (TS) Test (FIG. 4), all compared to their wild-type (WT) counterparts. We have further found that conditional, acute ablation of GI epithelial-derived SERT in development/adulthood (with our novel SERT^{FL/FL}/villin^{cre} and SERT_{FL/FL}/villin^{cre}-ERT2 mice), leads to anti-anxiety phenotypes in the Open Field, Novelty-Suppressed Feeding and elevated plus maze, open field and novelty-suppressed feeding tests (FIGS. 6-8), demonstrating that acute ablation of epithelial SERT in adulthood could potentially be an effective remedy for anxiety and depression. Importantly, the SERT^{FL/FL}/villin^{cre}-ERT2 mice also experienced a decreased visceromotor response to colorectal distention, indicating that acute ablation/blockade of epithelial SERT may also treat/prevent abdominal pain (FIG. 10). Abdominal pain is a highly undesirable side effect of SSRIs. Further, individuals who take SSRIs for anxiety or depression often have co-morbid abdominal pain associated disorders (functional GI disorders/disorders of gut-brain interactions). Conversely, mice that had selective deletion of SERT in the ENS, but not in the CNS or the intestinal epithelium (Wnt-1cre::SERT^{FL/FL} mice), displayed anxiogenic-like phenotypes in OF and NSF Tests (FIGS. 1-3) and increased behavioral despair in a test assessing for Learned Helplessness (LH) (FIG. 4).

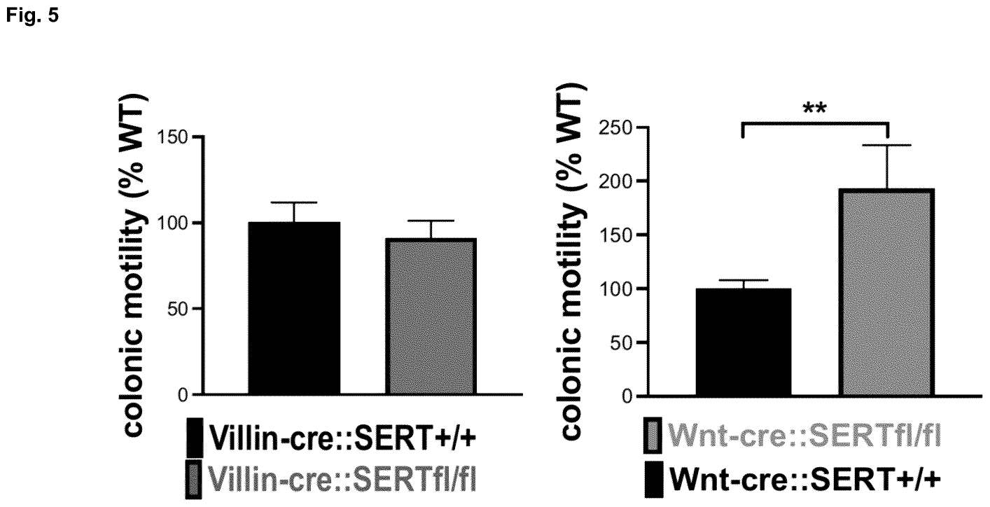

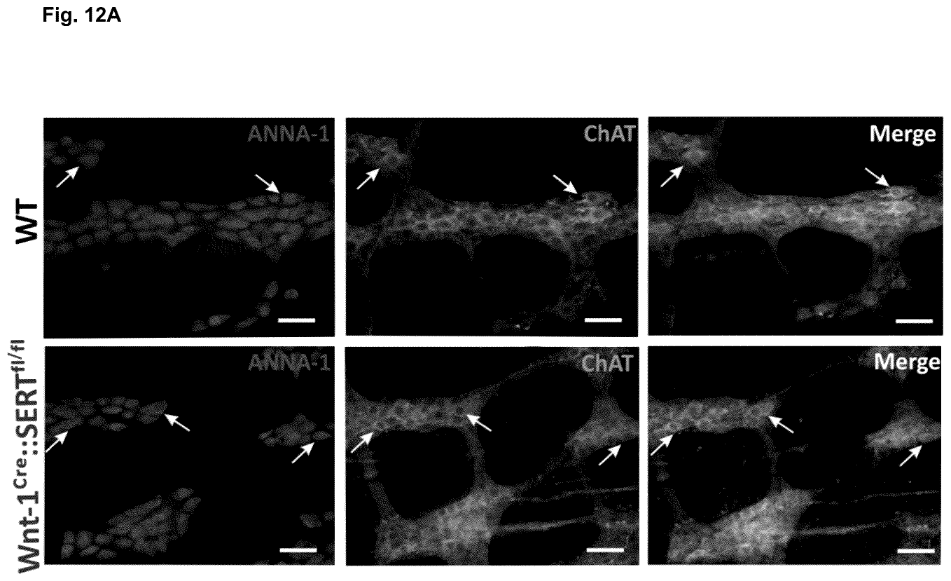

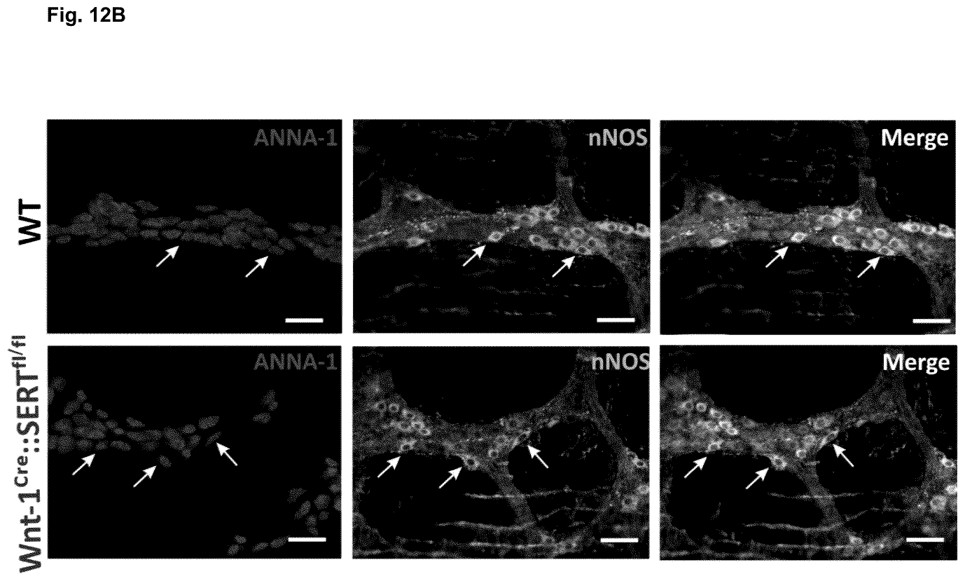

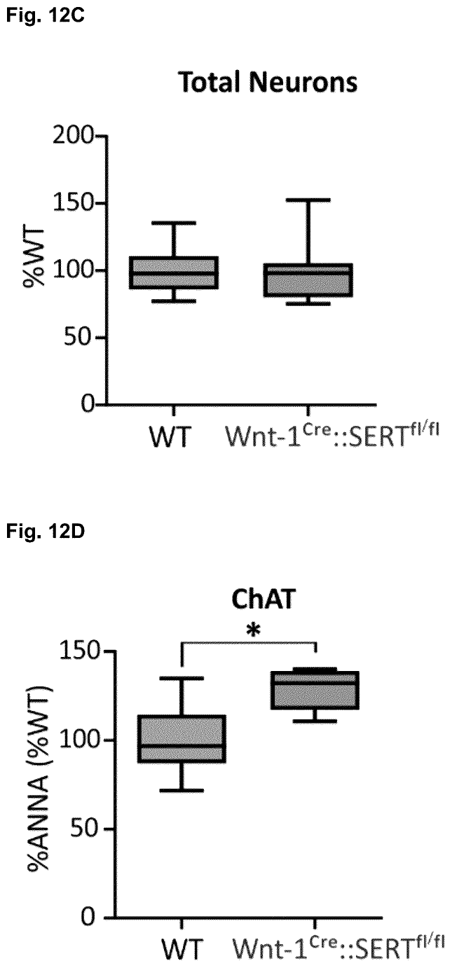

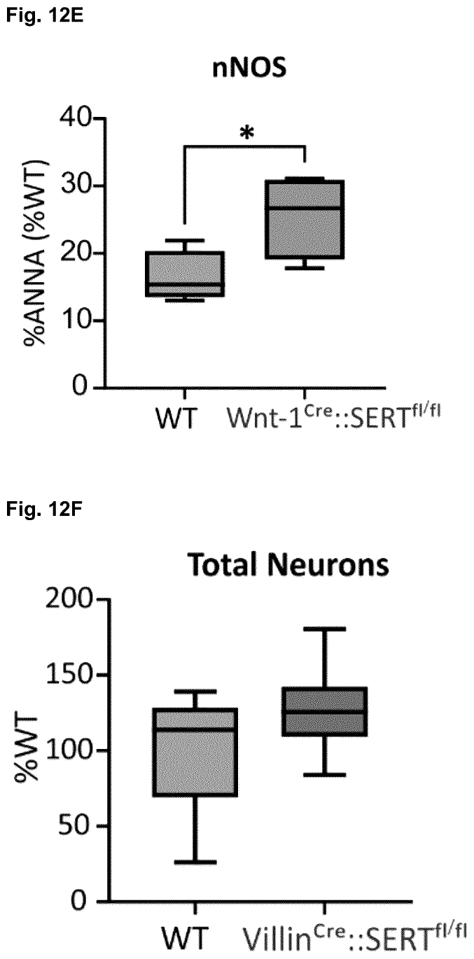

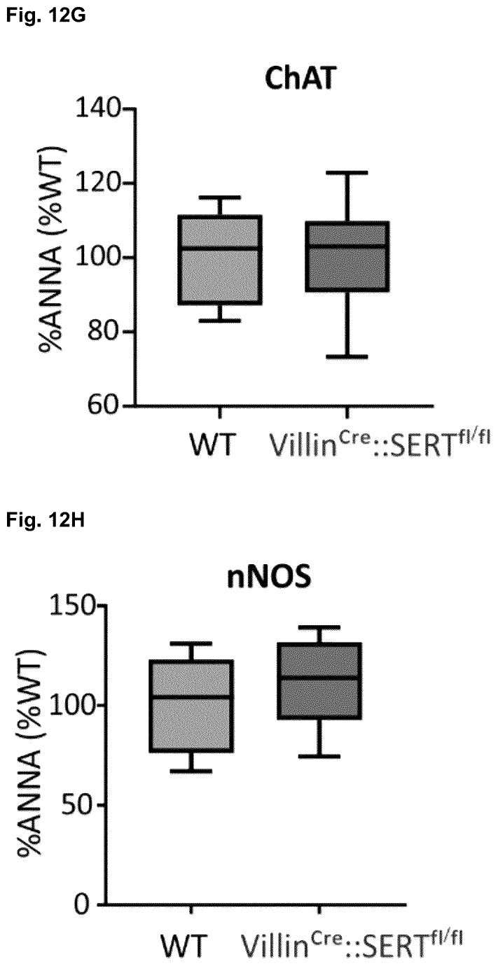

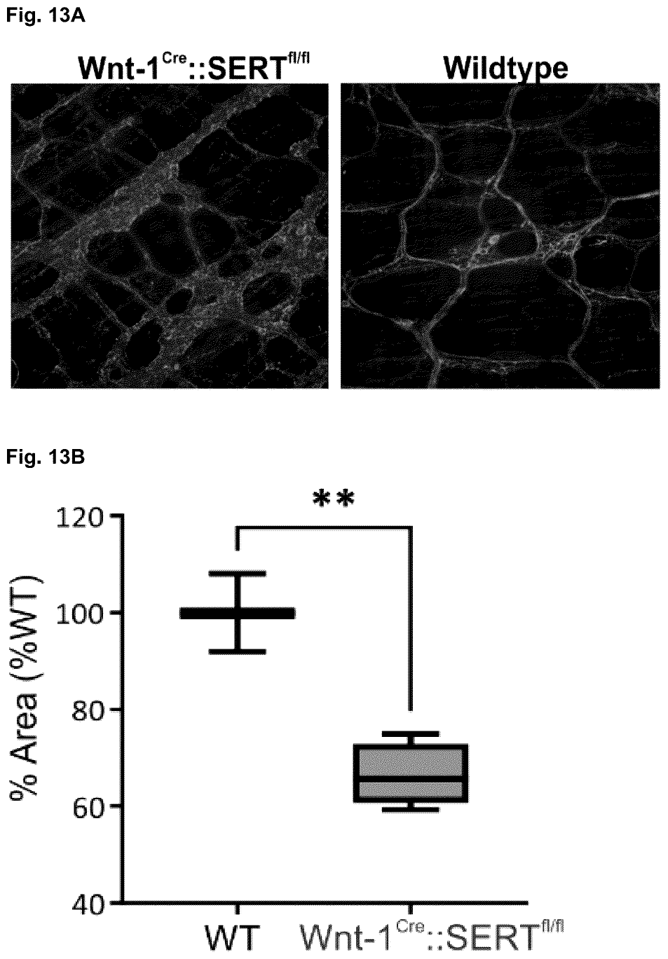

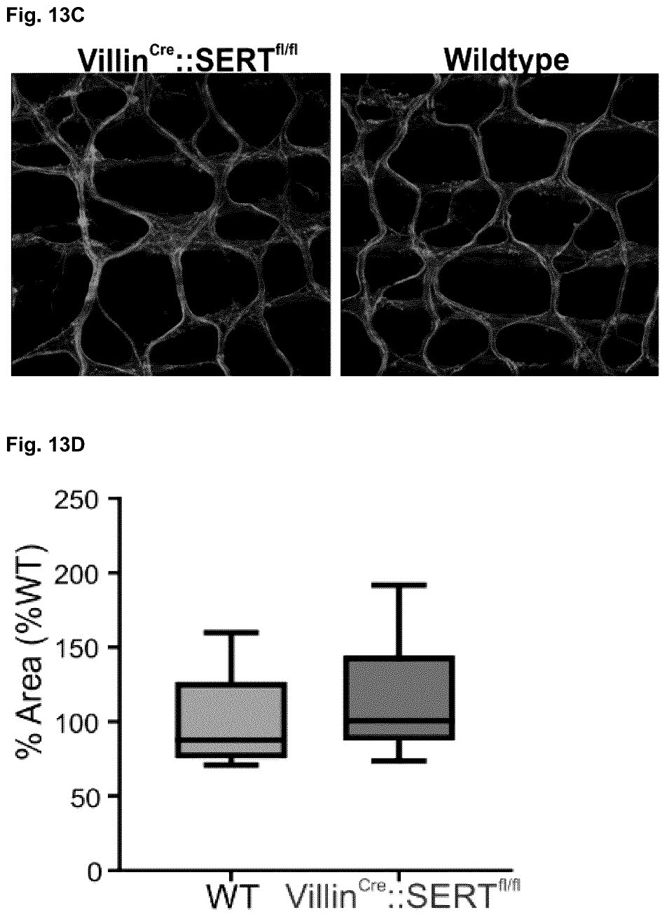

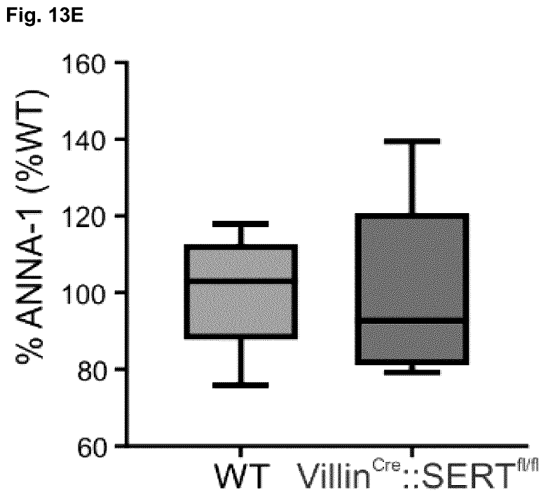

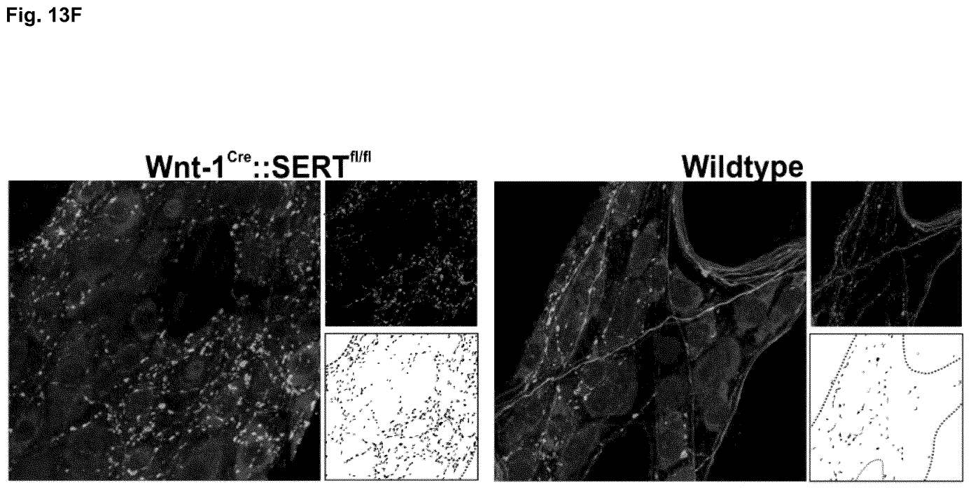

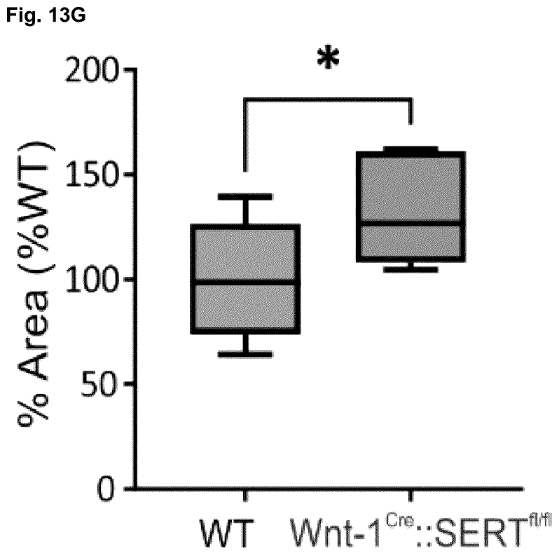

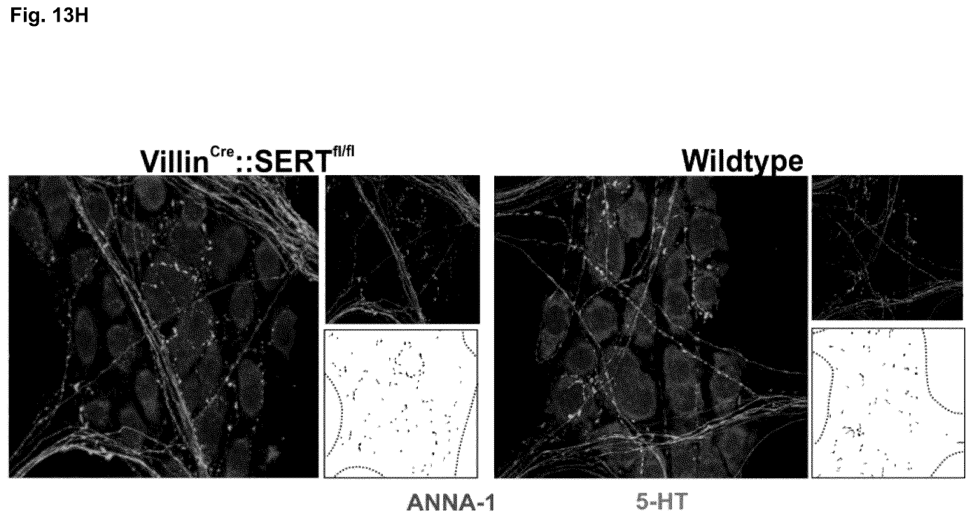

Furthermore, both lines also differed in their intestinal motility and enteric nervous system development phenotypes. The villin^{cre} mice:: SERT^{FL/FL} mice did not demonstrate any abnormalities in in vivo motility (FIG. 5) or morphological changes in total enteric neuron counts nor subsets of cholinergic, nitrergic or serotonergic neurons (FIGS. 12A-12H and 13A-12I). It has been previously found that global SERT deletion, in SERT KO mice, or hyperefficiency, in SERT Ala56 mice, can impact enteric serotonergic neuron and enterochromaffin (EC) cell development. To investigate whether EC cells and/or serotonergic neurons are altered in Wnt-1^{Cre}::SERT^{fl/fl} and Villin^{Cre}::SERT^{fl/fl} mice, we first examined 5-HT axonal projections in the MP of the colons of Wnt-1^{Cre}::SERT^{fl/fl} and Villin^{Cre}::SERT^{fl/fl} mice (FIGS. 13A and 13C). In the MP of Villin^{Cre}::SERT^{fl/fl} mice, 5-HT axonal projections (FIGS. 13B and 13D) and the proportion of 5-HT neurons (FIG. 13E) were not significantly different compared to WT. In Wnt-1^{Cre}::SERT^{fl/fl} mice, however, the distribution of 5-HT in neurons and their axons was highly abnormal (FIG. 13A). At the low power objective, serotonergic axonal projections were significantly reduced in the MP of Wnt-1^{Cre}::SERT^{fl/fl} mice compared to WT mice (FIG. 13B) while the accumulation of 5-HT into neuronal cell bodies was undetected. This prompted further analysis of 5-HT at a high power objective to quantify its distribution within each ganglion (FIGS. 13F and 13G). Despite the decrease in serotonergic axonal projections, Wnt-1^{Cre}::SERT^{fl/fl} mice had significantly more 5-HT varicosities within the ganglion compared to WT (FIG. 13G). These differences were not observed in the Villin^{Cre}::SERT^{fl/fl} mice (FIGS. 13H and 13I).

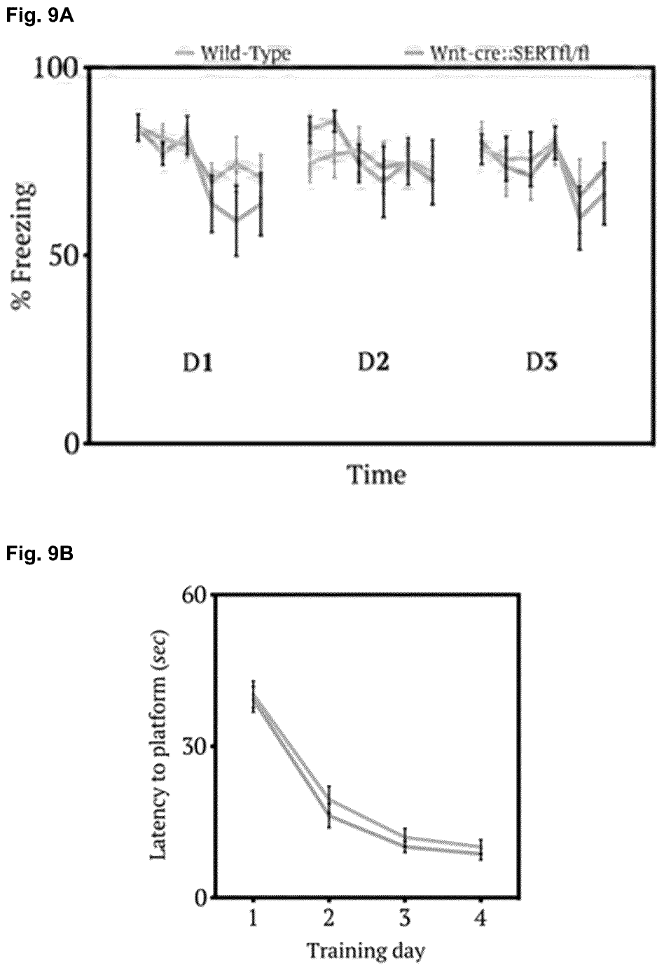

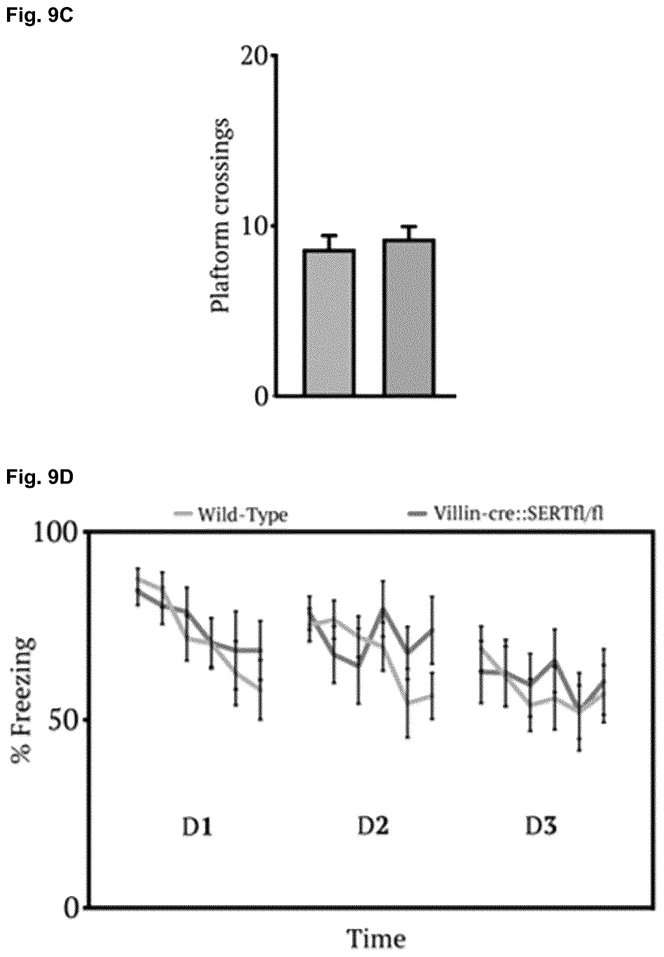

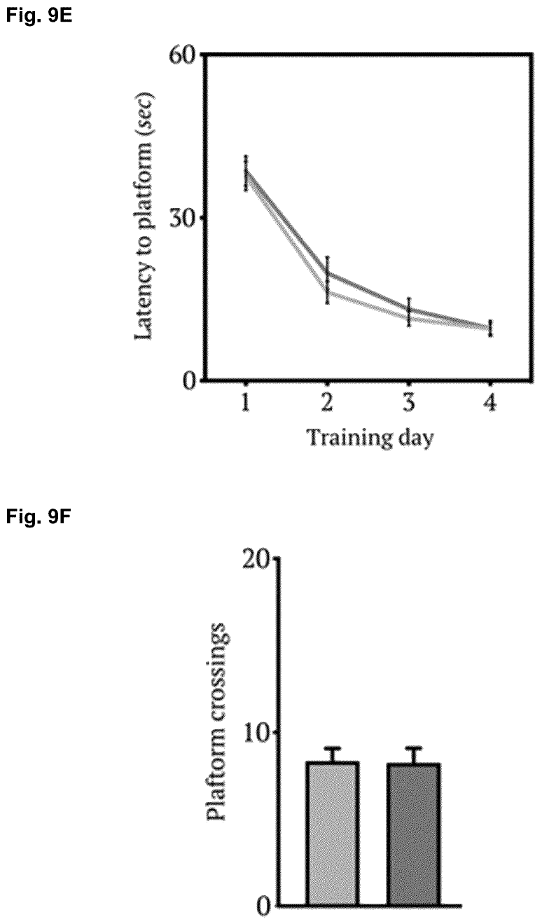

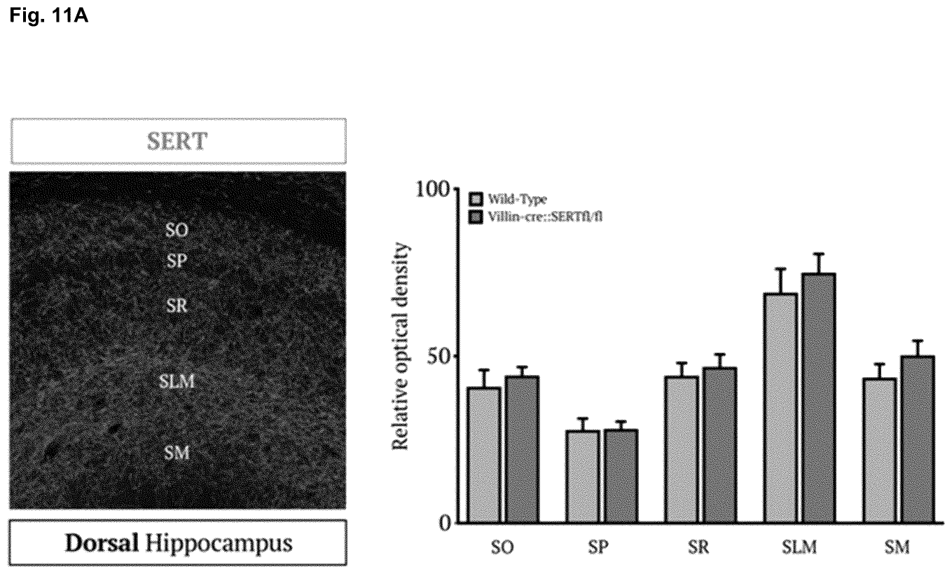

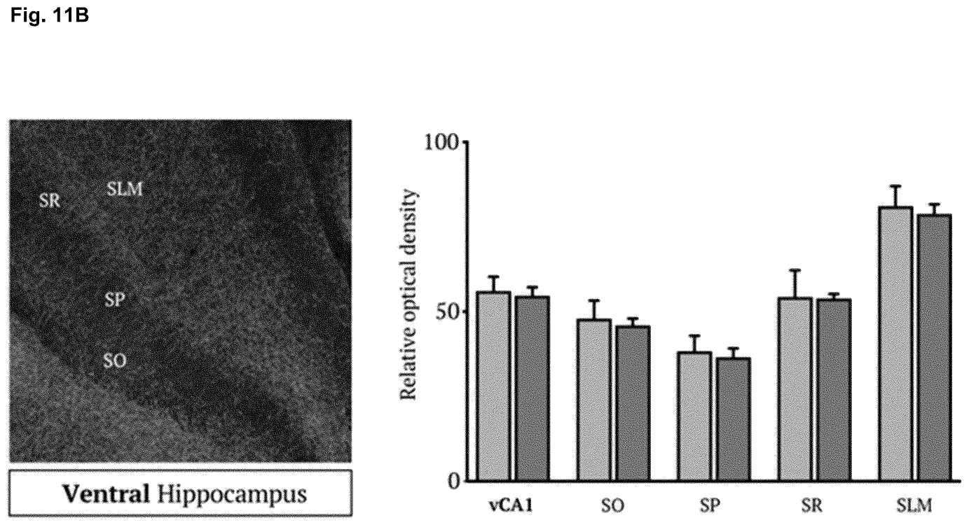

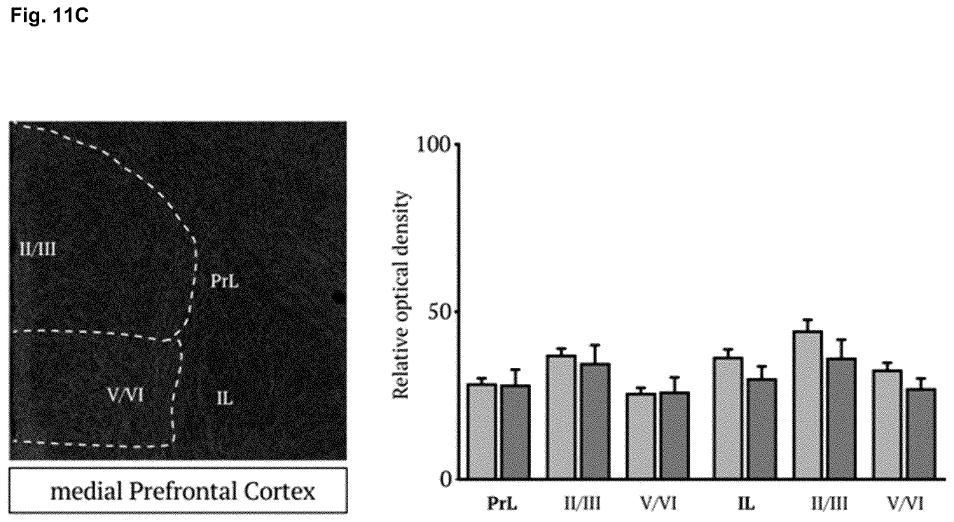

Importantly, there were also no abnormalities demonstrated in serotonergic pathways in the CNS in these mice (FIGS. 11A-11C) nor were there any differences in cognition (FIGS. 9A-9F), another potentially harmful side effect associated with in utero SSRI exposure. In contrast, the Wnt-1cre::SERT^{FL/FL} mice had significant slowed colonic motility (FIG. 5) and delays in gastric emptying (not shown) that were accompanied by distinct changes in cholinergic, nitergic and serotonergic enteric neuronal subsets, compared to their WT littermates (FIGS. 12A-12H and 13A-12I), which can be equated in patients to constipation and nausea, two side effects often accompanying SSRI intake (Margolis et al. 2016). Also importantly, the SERT^{FL/FL}/Wnt-1^{Cre} mice show increased sensitivity to colorectal distention (FIG. 10), suggesting that SERT inhibition below the gut mucosa increases sensitivity to abdominal pain.

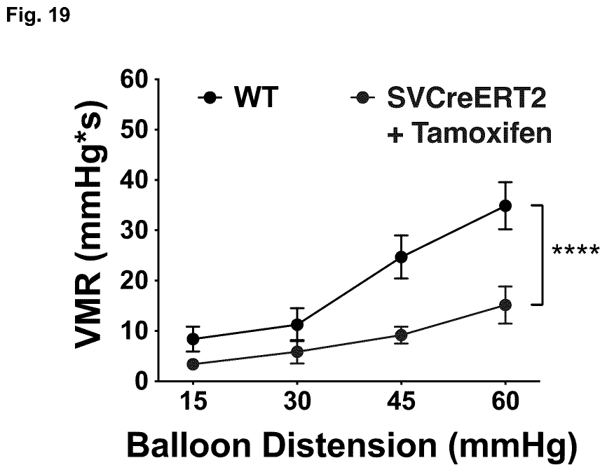

We also assessed for sensitivity to abdominal pain/intestinal nociception in SERTFL/FL/villinCre and the SERTFL/FL/Wnt-1Cre mice. Interestingly, we found that both strains of mice had increased sensitivity to visceral pain. Strikingly, however, when we tested inducible SERT-villin mice (SERTFL/FL/villin Cre-ERT2 mice) by eliminating SERT acutely during adulthood, we found that this affect was anti-nociceptive (FIG. 19), suggesting that inducing intestinal epithelial SERT blockade after development may be protective against intestinal pain/nociception and visceral hypersensitivity.

In summary, we found that (1) blockade of ENS SERT function causes anxiety and depression-like phenotypes, constipation and abdominal pain, suggesting that SSRI exposure to the ENS (that occurs with systemic absorption) can induce negative effects on mood (that SSRIs are designed to prevent) as well as GI function, all of which can cause discontinuation of current SSRI formulations; (2) Conversely, selective blockade of intestinal epithelial SERT function reduces anxiety and depression-like behaviors without causing in vivo GI dysmotility or abdominal pain, suggesting that SSRI exposure to the GI epithelium produces beneficial effects on mood without negatively impacting GI function; (3) In a separate study we showed that chronic systemic SSRI exposure acts on 5-HT2c receptors to inhibit CNS dopamine activity, which diminishes antidepressant and anxiolytic efficacy, showing that SSRI exposure to the CNS can have deleterious effects on mood.

Altogether, these data suggest that it may be possible to effectively treat anxiety and depression while simultaneously avoiding negative side effects (such as constipation and abdominal pain), and placental SSRI transfer during fetal development, by exclusive blockade of intestinal mucosal SERT. A non-absorbable SERT antagonist, that selectively antagonizes intestinal mucosal SERT, and thus amplifies epithelial 5-HT availability, while leaving systemic SERT (brain and ENS) unblocked, may thus be able to effectively treat mood disorders while avoiding deleterious effects (El Marroun et al. 2014). In the case of maternal SSRI use, such an agent would also critically decrease systemic bioavailability of SSRI to a developing fetus, thus limiting the potential effects of SSRIs on fetal neurodevelopment, and being a safer antidepressant alternative for pregnant women.

### Example 2

### A Gut Epithelial-Restricted Delivery System for SSRIs

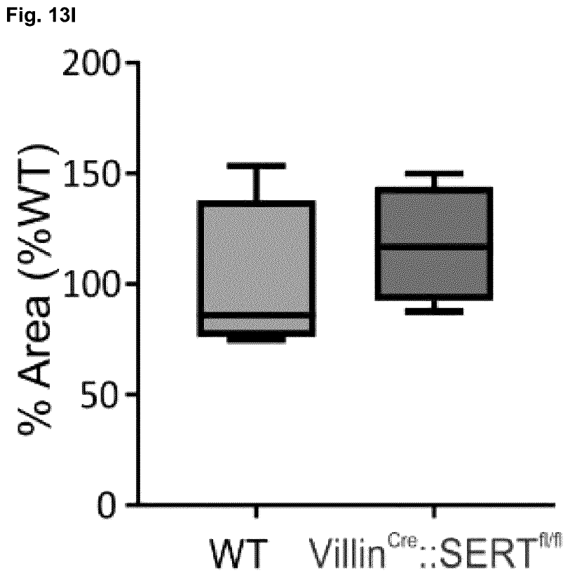

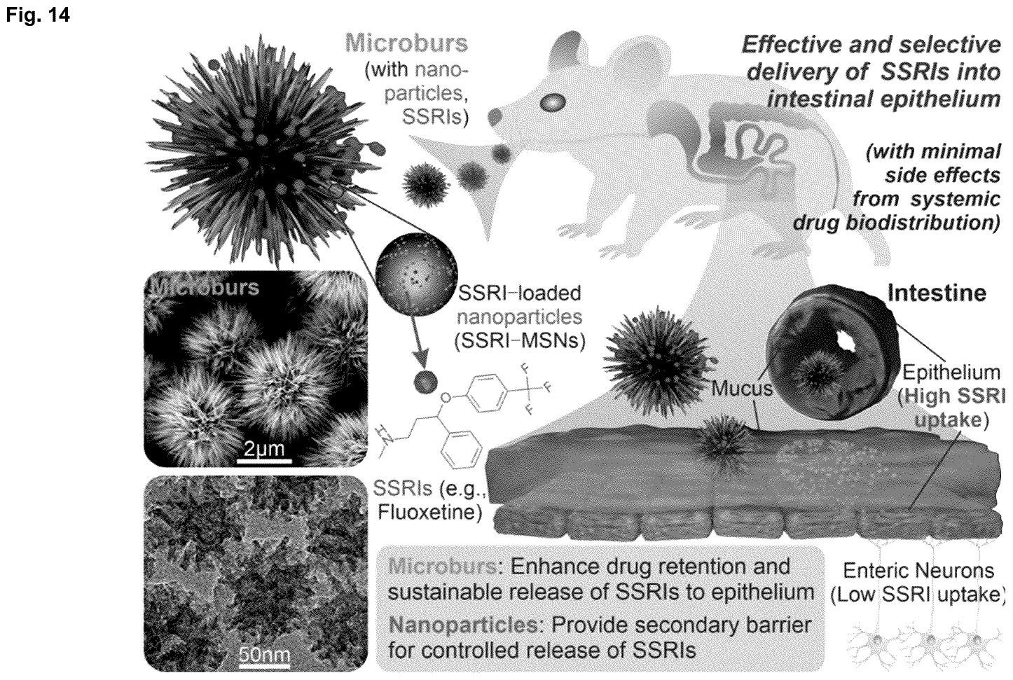

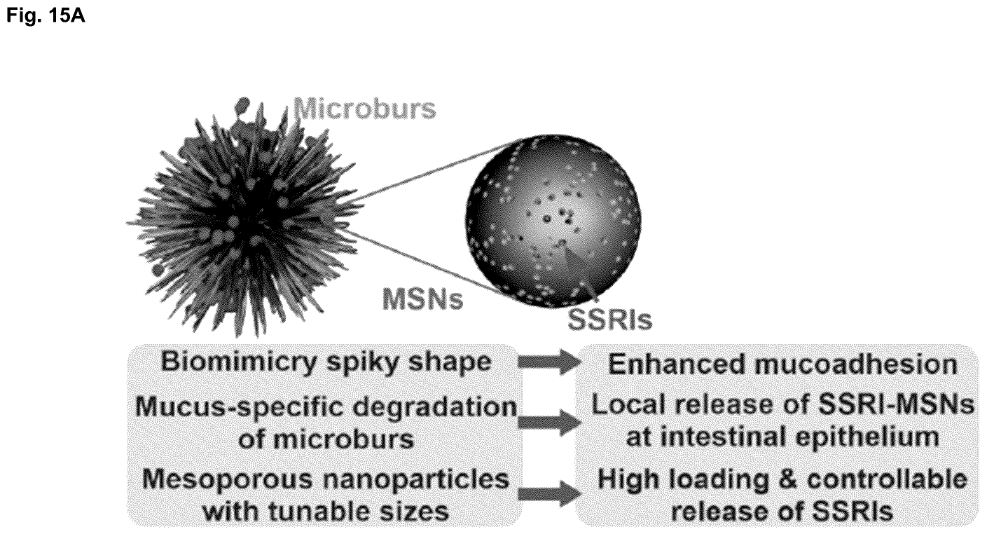

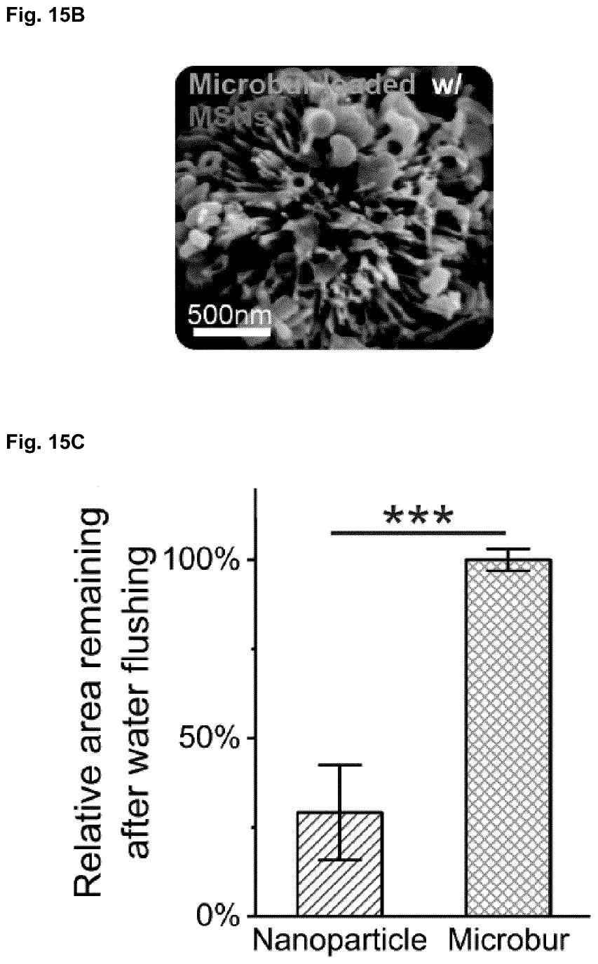

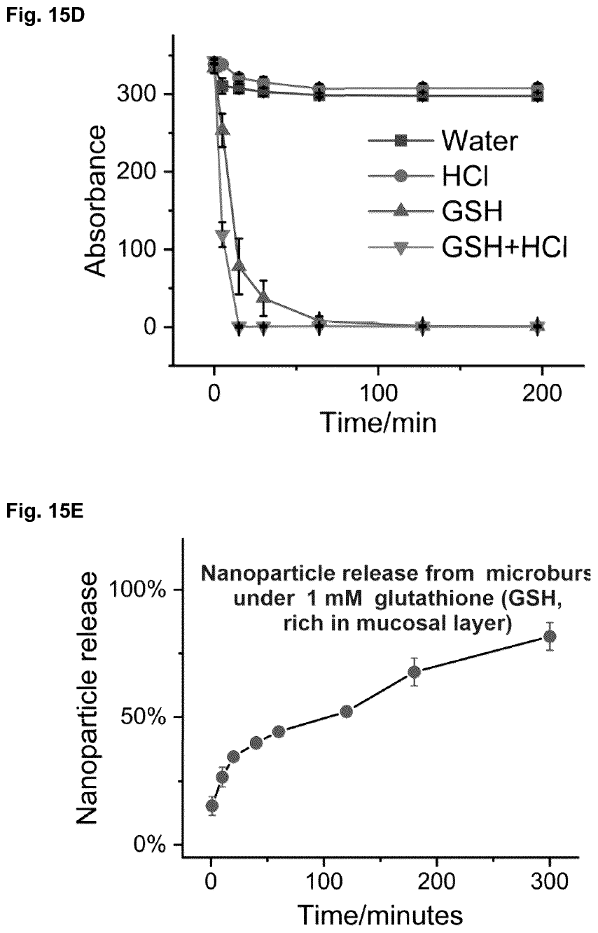

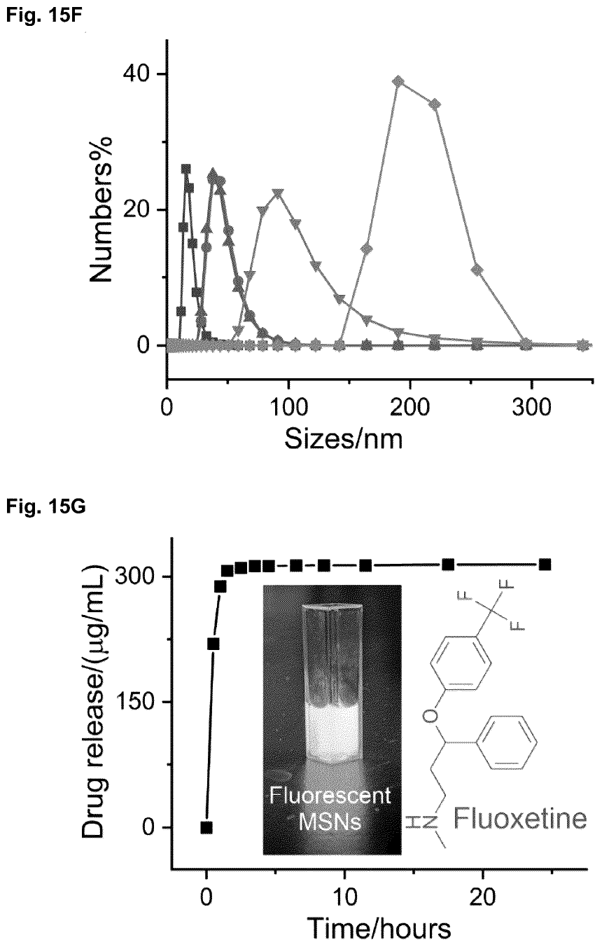

We have developed a 3D vertical nanoneedle-decorated microparticle-based delivery platform that adheres to the intestinal epithelium and can locally release SSRI-encapsulated nanoparticles in a controlled manner. This state-of-the-art 3D vertical nanoneedle-decorated microparticle represents a microscale mimicry of a spiky fruit bur (microbur, FIG. 14) that, when loaded with SSRI nanoparticles, enables local, controlled and sustained release (SR) of SSRIs to the intestinal epithelium while maximally blocking SSRI transfer below the gut epithelium and thus limiting systemic absorption. The enhanced, novel gut adherence technology (FIG. 15C) can simultaneously serve to enhance oral delivery efficiency.

There are multiple limitations to the currently available drug delivery systems that can enable SR of a drug restricted to the gut epithelium: (1) drugs that are not absorbed by the epithelium are eradicated quickly through the GI tract by peristalsis. In contrast, the 3D vertical nanoneedle-decorated microparticle design disclosed herein enables the device to stay within the gut epithelium despite gut contractions; (2) a microneedle-based oral drug delivery platform has been developed that can enhance GI adhesion but has larger microneedle sizes cause damage to the GI epithelium, which promote translocation (and thus systemic absorption) of drug (Sie et al. 2012); (3) nanoneedle-based transdermic delivery patches have been invented but cannot be adapted for oral delivery. The biocompatible, 3D vertical nanoneedle-decorated microparticle-based delivery platform disclosed herein addresses these critical issues, and is thus much more likely to facilitate the safe, efficient, convenient, and reliable oral delivery of SSRIs to the gut epithelium.

For epithelial-restricted targeting of SSRIs, we utilize a hollow manganese (Mn) dioxide microbur loaded with SSRI encapsulated mesoporous silica nanoparticles (SSRI-MSNs, FIGS. 14 and 15A-15G). Both components have known biocompatibility, paving the way for the translation of this proposed technology (Tanoshima et al. 2014). The microbur sizes range from 1-5 μm; they do not penetrate the mucosal layer yet robustly adhere to it which significantly enhances their retention in the GI tract (FIG. 15C). The microbur is also highly stable in acidic conditions, allowing it to bypass the stomach (FIG. 15D). In contrast, it will degrade in the mucus-rich glutathione (˜1 mM) of the intestine in a controllable manner (from a hours to days), thereby initiating the releasing the SSRI-MSNs within one cycle of intestinal mucus turnover (several hours; FIGS. 15D and 15E). Further, the release of Mn ions associated with the degradation could result in the activation of Ca²⁺-dependent adhesion molecules (cadherin), or directly upregulate the expression of tight junction-related genes (e.g., Claudin or occludin), thereby enhancing the integrity of epithelial tight junctions (McVey et al. 2019). As such, the degradation product may further reduce the diffusion of SSRI into the bloodstream, and thus systemic absorption, by enhancing the integrity of gut epithelial tight junctions. In parallel, MSNs, with tunable sizes and internal pore diameters, provide a secondary barrier to restrict drug diffusion and facilitate local sustainable release of SSRI (FIG. 15F). MSNs covalently doped with fluorescent dyes (e.g., fluorescein isothiocyanate) have been loaded with SSRIs to visualize the distribution of SSRI-MSNs and confirm their selective uptake by intestinal epithelium through ex vivo imaging (FIG. 15G). The SSRI-MSNs can be further enhanced to reduce the diffusion of the nanoparticles across the mucosal layer, either through conjugation of polyethylene glycols (PEG) or by optimizing their sizes (from 10 to >400 nm). In these ways, the delivery platform facilitates efficient, selective, and visualizable oral delivery of SSRIs to intestinal epithelial cells, where SERT in the intestinal mucosa is primarily located. The microscale devices also efficiently (e.g., >50% of total weight) and rapidly (e.g., within 5 minutes) adhere to the mucus of the small or large intestine, and drug release can be targeted to the different parts of the GI tract based on pH or biological stimuli (e.g., intestine-specific enzymes). After adhesion to the mucus, the microscale devices release SSRI into the mucosal layer of the intestine and stay within the epithelium, allowing targeted SSRI blockade to the mucosa with minimal diffusion across the epithelial barrier to, ultimately, decrease systemic distribution.

To further enhance the targeted delivery into the intestinal epithelium, nanoparticles encapsulating SSRIs can be functionalized with epithelial-targeting ligands (e.g., lactoferrin) loaded to the microscale device. This targeted technology may thus provide a novel way to treat anxiety and depression and simultaneously, by minimizing systemic SSRI diffusion (e.g. to blood, ENS, CNS, placenta), decrease the likelihood for deleterious side effects and placental transfer to a fetus.

### Example 3

### Preparation of the MnO₂ bio-microburs

To prepare bio-microburs with a composition of doped or un-doped MnO₂, in 50 mL plastic centrifuge tubes, aqueous solutions of Mn(CH₃COOH)₂·4H₂O were first prepared at a concentration of 3.65 g per 200 mL and (NH₄)₂S₂O₈ at a concentration of 3.90 g per 200 mL, which was enough for 10 reactions (40 mL total volume for each reaction). 20 mL of the Mn(CH₃COOH)₂·4H₂O aqueous solution was added to 20 mL of (NH₄)₂S₂O₈ drop by drop under vigorous stirring at 1200 rpm for 10-20 minutes until the solution became pale yellow. 1.6 mL of concentrated sulfuric acid (H₂SO₄, 95-98%) was added to the yellow solution. The solution continued to stir for 10 minutes. The solution was transferred to a 40 mL hydrothermal chamber and heated from room temperature to 120° C. within 30 minutes, followed by continuous heating at 120° C. for 5 hours. The MnO₂ bio-microburs (black-colored precipitates) were washed with water and ethanol two times each, until the pH became neutral (pH=7). The bio-microburs were washed using freeze-drying instruments. To dope the bio-microburs with different amounts of silver, aluminum, iron, and other ions, the nitrate salts with varying amounts were added in the first step, together with the Mn(CH₃COOH)₂·4H₂O. To modulate the size, shape, and structure of the bio-microburs, concentrations of the precursors in the first step, and the temperature and heating times were varied.

To coat the bio-microburs with a biopolymer (e.g., a chitosan coating), chitosan was prepared as a stock solution. For example, 100 mL 1% acetic acid DIW solution was prepared in a 500 mL beaker, and 5 g of chitosan powder was added into the acetic acid DIW solution. A magnetic stirring bar was added and the mixture was stirred vigorously. The mixture became very viscous and sticky, so the stirring speed was adjusted accordingly. Stirring occurred for 12-36 hours, and the solution became gel-like. The gel-like solution was transferred into 50 mL centrifuge tubes and stored properly. There was less than 50 mL of solution that could be transferred from the original 100 mL solution. 1-2 mL gel-like chitosan solution was transferred by directly pouring it into a 50 mL clean beaker with a stirring bar inside. The amount of chitosan solution was estimated based on the weight of the beaker, before and after the transfer. 5-10 mL of water was added into the beaker to make a 1-10 mg/mL chitosan solution by stirring. 1-50 mg of bio-microbur powder was placed in a 50 mL centrifuge tube, DIW (10-20 mL) was added, and this was followed by vigorously shaking/vortexing to break the powder into individual microparticles and form a bio-microbur suspension. The bio-microbur suspension was slowly added (shaking the suspension each time before adding) into 5 mL of the 10 mg/mL chitosan solution with vigorous stirring. The reaction took approximately 12 hours, then 1-10 mL 1× or 10×PBS was added slowly, and the reaction was continued for another 12 hours. When 10 mg/mL chitosan solution was used, the solution became cloudy due to the precipitation of chitosan. Washing with DIW was performed by centrifugation or gravity precipitation, keeping the aggregates in the bottom of centrifuge tubes. Washing was repeated 3-6 times, and the bio-microburs were maintained in DIW or buffer conditions (being careful to not let it dry).

### Example 4

### Sustained Gut Retention, Pharmacokinetics and Biosafety of the Novel Delivery System

The novel 3D vertical nanoneedle-decorated microparticle-based delivery platform that remains stable within the intestinal epithelium and can release nanoparticle-filled SSRIs in controlled, gut epithelium-restricted fashion. The microscale mimicry of spiky fruit bur (FIG. 14) that, loaded with SSRI nanoparticles, enables controlled release SSRIs while maximally blocking SSRI transfer below the gut epithelium (in vitro and in vivo) and thus ENS/systemic absorption.

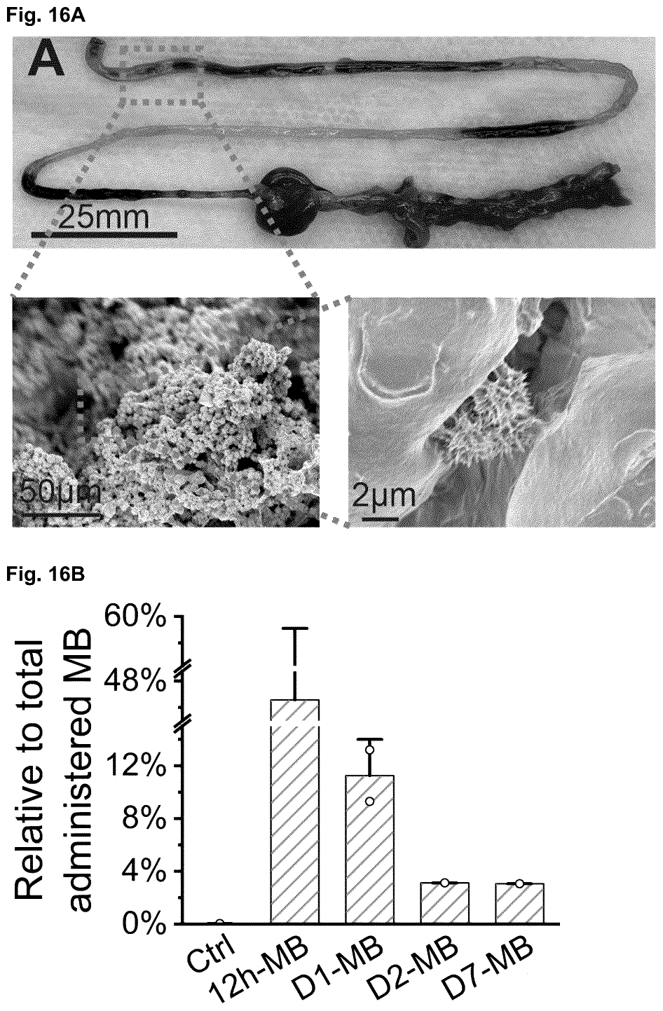

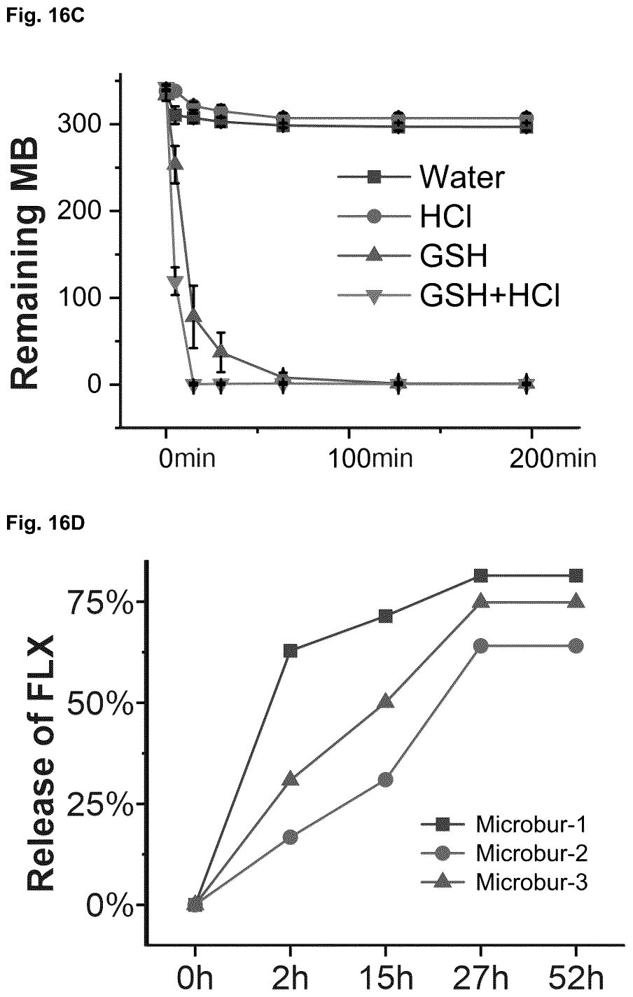

Sustained gut retention of microburs (MBs) was observed on the intestinal epithelium, which allows for a sustained release (SR) capacity and thus sustained effect on the target area (FIGS. 16A and 16B). The microburs also exhibited excellent stability in acidic conditions and biodegradability in mucus glutathione (GSH) (FIG. 16C), indicating that the MB can survive transit through the highly acidic stomach to disperse in its goal location of the intestine. Furthermore, modulation of sustainable fluoxetine (FLX) release profile from MB with different structures demonstrated our ability to change the size of the MBs in order to regulate SSRI dose/exposure for maximal efficacy (FIG. 16D).

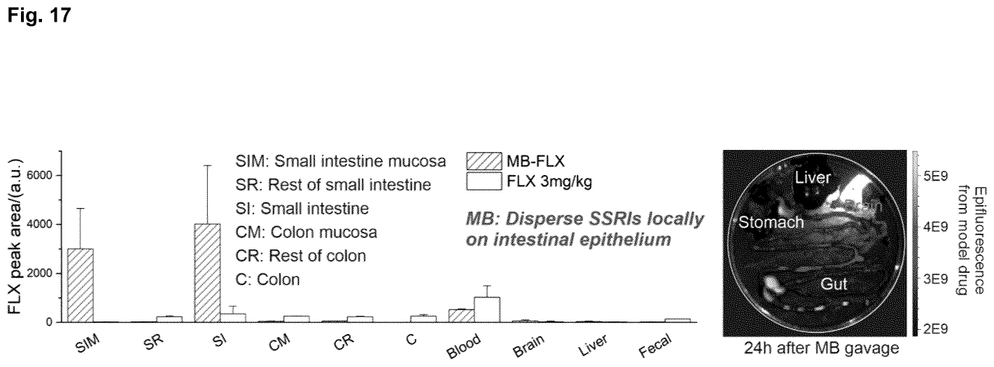

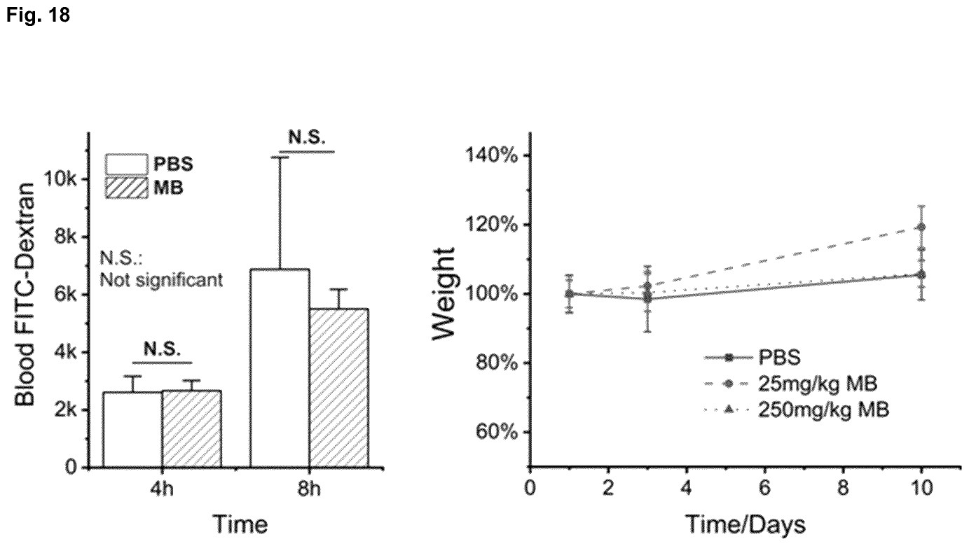

In vivo FLX pharmacokinetic study illustrated the local dispersion of FLX on small intestine epithelium with minimal systemic biodistribution after 12-hours of FLX release from microburs (FIG. 17, left). In vivo imaging system analysis of model drug (Fluorescein) distribution after delivery by MBs also supported the selective dispersion of drug in the gut (FIG. 17, right).

The microburs delivery system are also biologically safe, with no increased epithelial permeability (FIG. 18, left) nor weight loss (FIG. 18, right) observed after repeated oral gavage of mice with MB-MSN-SSRIs with doses of fluoxetine (FLX)>10-fold greater than standard dosing (250 mg/kg). A further advantage of this technology is that we have further combined the microburs with nanoparticle-mediated biologic delivery to further enhance oral delivery efficiency.

### DOCUMENTS CITED

1. Brody D J, Pratt L A, Hughes J P. Prevalence of Depression Among Adults Aged 20 and Over: United States, 2013-2016. NCHS Data Brief 2018:1-8.
2. https://www.hep.med.harvard.edu/ncs/index.php. Harvard Medical School, 2007. National Comorbidity Survey (NCS). 2007.
3. Twenge J M, Joiner T E. U.S. Census Bureau-assessed prevalence of anxiety and depressive symptoms in 2019 and during the 2020 COVID-19 pandemic. Depress Anxiety 2020; 37:954-956.
4. Thibaut F. Anxiety disorders: a review of current literature. Dialogues Clin Neurosci 2017; 19:87-88.
5. Jacobsen J P R, Krystal A D, Krishnan K R R, et al. Adjunctive 5-Hydroxytryptophan Slow-Release for Treatment-Resistant Depression: Clinical and Preclinical Rationale. Trends Pharmacol Sci 2016; 37:933-944.
6. Trivedi M H, Rush A J, Wisniewski S R, et al. Evaluation of outcomes with citalopram for depression using measurement-based care in STAR*D: implications for clinical practice. Am J Psychiatry 2006; 163:28-40.
7 Gollan J K, Fava M, Kurian B, et al. What are the clinical implications of new onset or worsening anxiety during the first two weeks of SSRI treatment for depression? Depress Anxiety 2012; 29:94-101.
8. Sinclair L I, Christmas D M, Hood S D, et al. Antidepressant-induced jitteriness/anxiety syndrome: systematic review. Br J Psychiatry 2009; 194:483-90.
9. Bassotti G. Motions and emotions: the treatment of depression causes constipation. Neurogastroenterol Motil 2000; 12:113-5.
10. Marken P A, Munro J S. Selecting a Selective Serotonin Reuptake Inhibitor: Clinically Important Distinguishing Features. Prim Care Companion J Clin Psychiatry 2000; 2:205-210.
11. Al-Jumah K A, Qureshi N A. Impact of pharmacist interventions on patients' adherence to antidepressants and patient-reported outcomes: a systematic review. Patient Prefer Adherence 2012; 6:87-100.
12. Gaynes B N, Gavin N, Meltzer-Brody S, et al. Perinatal depression: prevalence, screening accuracy, and screening outcomes. Evid Rep Technol Assess (Summ) 2005:1-8.
13. Alwan S, Friedman J M, Chambers C. Safety of Selective Serotonin Reuptake Inhibitors in Pregnancy: A Review of Current Evidence. CNS Drugs 2016.
14. Malm H, Brown A S, Gissler M, et al. Gestational Exposure to Selective Serotonin Reuptake Inhibitors and Offspring Psychiatric Disorders: A National Register-Based Study. J Am Acad Child Adolesc Psychiatry 2016; 55:359-66.
15. Apter-Levy Y, Feldman M, Vakart A, et al. Impact of maternal depression across the first 6 years of life on the child's mental health, social engagement, and empathy: The moderating role of oxytocin. Am J Psychiatry 2013; 170:1161-8.
16. O'Connor T G, Monk C, Burke A S. Maternal Affective Illness in the Perinatal Period and Child Development: Findings on Developmental Timing, Mechanisms, and Intervention. Curr Psychiatry Rep 2016; 18:24.
17. Yonkers K A, Blackwell K A, Glover J, et al. Antidepressant use in pregnant and postpartum women. Annu Rev Clin Psychol 2014; 10:369-92.
18. Andrade S E, Raebel M A, Brown J, et al. Use of antidepressant medications during pregnancy: a multisite study. Am J Obstet Gynecol 2008; 198:194 e1-5.
19. Gentile S. SSRIs in pregnancy and lactation: emphasis on neurodevelopmental outcome. CNS Drugs 2005; 19:623-33.
20. Gentile S. The safety of newer antidepressants in pregnancy and breastfeeding. Drug Saf 2005; 28:137-52.
21. Hendrick V, Stowe Z N, Altshuler L L, et al. Placental passage of antidepressant medications. Am J Psychiatry 2003; 160:993-6.
22. Nijenhuis C M, Horst P G, Berg L T, et al. Disturbed development of the enteric nervous system after in utero exposure of selective serotonin re-uptake inhibitors and tricyclic antidepressants. Part 1: Literature review. Br J Clin Pharmacol 2012; 73:16-26.
23. Nijenhuis C M, ter Horst P G, van Rein N, et al. Disturbed development of the enteric nervous system after in utero exposure of selective serotonin re-uptake inhibitors and tricyclic antidepressants. Part 2: Testing the hypotheses. Br J Clin Pharmacol 2012; 73:126-34.
24. Ansorge M S, Morelli E, Gingrich J A. Inhibition of serotonin but not norepinephrine transport during development produces delayed, persistent perturbations of emotional behaviors in mice. J Neurosci 2008; 28:199-207.
25. Ansorge M S, Zhou M, Lira A, et al. Early-life blockade of the 5-H T transporter alters emotional behavior in adult mice. Science 2004; 306:879-81.
26. Margolis K G, Li Z, Stevanovic K, et al. Serotonin transporter variant drives preventable gastrointestinal abnormalities in development and function. J Clin Invest 2016; 126:2221-35.
27. El Marroun H, White T, Verhulst F C, et al. Maternal use of antidepressant or anxiolytic medication during pregnancy and childhood neurodevelopmental outcomes: a systematic review. Eur Child Adolesc Psychiatry 2014; 23:973-92.
28. Sie S D, Wennink J M, van Driel J J, et al. Maternal use of SSRIs, SNRIs and NaSSAs: practical recommendations during pregnancy and lactation. Arch Dis Child Fetal Neonatal Ed 2012; 97:F472-6.
29. Tanoshima R, Bournissen F G, Tanigawara Y, et al. Population P K modelling and simulation based on fluoxetine and norfluoxetine concentrations in milk: a milk concentration-based prediction model. Br J Clin Pharmacol 2014; 78:918-28.
30. McVey Neufeld K A, Bienenstock J, Bharwani A, et al. Oral selective serotonin reuptake inhibitors activate vagus nerve dependent gut-brain signalling. Sci Rep 2019; 9:14290.

All patents, patent applications, and publications cited herein are incorporated herein by reference in their entirety as if recited in full herein.

The disclosure being thus described, it will be obvious that the same may be varied in many ways. Such variations are not to be regarded as a departure from the spirit and scope of the disclosure and all such modifications are intended to be included within the scope of the following claims.

What is claimed is:

1. A method for treating or ameliorating the effect of a disorder in a subject, comprising administering to the subject an effective amount of an agent that selectively antagonizes intestinal mucosal serotonin reuptake transporter (SERT) with limited or no passage through the intestinal epithelial barrier.

2. The method of claim 1, wherein the agent has limited or no effect on SERT in central nervous system (CNS) and enteric nervous system (ENS).

3. The method of claim 1, wherein the disorder is selected from the group consisting of: a gastrointestinal disorder, a central nervous system disorder, an anxiety disorder, a mood disorder, a depressive disorder, an autism spectrum disorder, a substance abuse or dependence disorder, an attention deficit hyperactivity disorder (ADHD), a post-traumatic stress disorder (PTSD), and combinations thereof.

4. The method of claim 3, wherein the gastrointestinal disorder is selected from the group consisting of: abdominal pain, constipation, nausea, intestinal inflammatory disease, disorders of gut-brain interactions (e.g., irritable bowel syndrome), enteric nervous system hyperplasia, Crohn's disease, ulcerative colitis, microscopic colitis, and combinations thereof.

5. The method of claim 4, wherein the gastrointestinal disorder is abdominal pain.

6. The method of claim 1, wherein the agent is selected from the group consisting of: a selective serotonin reuptake inhibitor (SSRI), a serotonin-norepinephrine reuptake inhibitor (SNRI), a tricyclic antidepressant (TCA), an atypical antidepressant, and combinations thereof.

7. The method of claim 6, wherein the agent is an SSRI.

8. The method of claim 1, wherein the subject is a mammal.

9. The method of claim 8, wherein the subject is human.

10. The method of claim 9, wherein the subject is pregnant.

11. The method of claim 1, wherein the selective antagonism of intestinal mucosal SERT is achieved with assistance of a gut epithelial-restricted delivery system.

12. The method of claim 11, wherein the gut epithelial-restricted delivery system is a bio-microbur therapeutic delivery platform comprising: a spherical, hollow core having a surface, and a plurality of nanoneedles secured to the surface of the core and extending outwardly therefrom.

13. The method of claim 12, wherein the core and nanoneedles comprise manganese oxide (MnO₂) or titanium oxide (TiO₂).

14. The method of claim 12, wherein the core is loaded inside with SSRI encapsulated mesoporous silica nanoparticles.

15. The method of claim 12, wherein the nanoneedles have an average length of about 1 nm to 100 nm.

16. The method of claim 12, wherein the bio-micorbur has a size in the range of 1 μm to 5 μm.

17. A method for treating or ameliorating the effect of a disorder in a pregnant subject while preventing a negative effect on the fetus, comprising administering to the pregnant subject an effective amount of an agent that selectively antagonizes intestinal mucosal serotonin reuptake transporter (SERT) with limited or no passage through the intestinal epithelial barrier.

18. The method of claim 17, wherein the agent has limited or no effect on SERT in central nervous system (CNS) and enteric nervous system (ENS).

19. The method of claim 17, wherein the disorder is selected from the group consisting of: a gastrointestinal disorder, a central nervous system disorder, an anxiety disorder, a mood disorder, a depressive disorder, an autism spectrum disorder, a substance abuse or dependence disorder, an attention deficit hyperactivity disorder, a post-traumatic stress disorder (PTSD), and combinations thereof.

20. The method of claim 19, wherein the gastrointestinal disorder is selected from the group consisting of: abdominal pain, constipation, nausea, intestinal inflammatory disease, disorders of gut-brain interactions (e.g., irritable bowel syndrome), enteric nervous system hyperplasia, Crohn's disease, ulcerative colitis, microscopic colitis, and combinations thereof.

21. The method of claim 20, wherein the gastrointestinal disorder is abdominal pain.

22. The method of claim 17, wherein the agent is selected from the group consisting of: a selective serotonin reuptake inhibitor (SSRI), a serotonin-norepinephrine reuptake inhibitor (SNRI), a tricyclic antidepressant (TCA), an atypical antidepressant, and combinations thereof.

23. The method of claim 22, wherein the agent is an SSRI.

24. The method of claim 17, wherein the negative effect on the fetus is selected from the group consisting of deficit gut and/or brain neurodevelopment/function, attention deficit hyperactivity disorder (ADHD), anxiety, depression, decreased cognitive and social functioning, gastrointestinal (GI) mobility disorder, and combinations thereof.

25. The method of claim 17, wherein the selective antagonism of intestinal mucosal SERT is achieved with assistance of a gut epithelial-restricted delivery system.

26. The method of claim 25, wherein the gut epithelial-restricted delivery system is a bio-microbur therapeutic delivery platform comprising: a spherical, hollow core having a surface, and a plurality of nanoneedles secured to the surface of the core and extending outwardly therefrom.

27. The method of claim 26, wherein the core and nanoneedles comprise manganese oxide (MnO₂) or titanium oxide (TiO₂).

28. The method of claim 26, wherein the core is loaded inside with SSRI encapsulated mesoporous silica nanoparticles.

29. The method of claim 26, wherein the nanoneedles have an average length of about 1 nm to 100 nm.

30. The method of claim 26, wherein the bio-microbur has a size in the range of 1 μm to 5 μm.

31. A composition for treating or ameliorating the effect of a disorder in a subject, the composition comprising a gut epithelial-restricted delivery system comprising a particle-based control release device and an agent disposed on a surface of the device, wherein the agent, upon release from the surface of the device, selectively antagonizes intestinal mucosal serotonin reuptake transporter (SERT) with limited or no passage through the intestinal epithelial barrier.

32. The composition of claim 31, wherein the agent has limited or no effect on SERT in central nervous system (CNS) and enteric nervous system (ENS).

33. The composition of claim 31, wherein the disorder is selected from the group consisting of: a gastrointestinal disorder, a central nervous system disorder, an anxiety disorder, a mood disorder, a depressive disorder, an autism spectrum disorder, a substance abuse or dependence disorder, an attention deficit hyperactivity disorder (ADHD), a post-traumatic stress disorder (PTSD), and combinations thereof.

34. The composition of claim 33, wherein the gastrointestinal disorder is selected from the group consisting of: abdominal pain, constipation, nausea, intestinal inflammatory disease, disorders of gut-brain interactions (e.g., irritable bowel syndrome), enteric nervous system hyperplasia, Crohn's disease, ulcerative colitis, microscopic colitis, and combinations thereof.

35. The composition of claim 34, wherein the gastrointestinal disorder is abdominal pain.

36. The composition of claim 31, wherein the agent is selected from the group consisting of: a selective serotonin reuptake inhibitor (SSRI), a serotonin-norepinephrine reuptake inhibitor (SNRI), a tricyclic antidepressant (TCA), an atypical antidepressant, and combinations thereof.

37. The composition of claim 36, wherein the agent is an SSRI.

38. The composition of claim 31, wherein the subject is a mammal.

39. The composition of claim 38, wherein the subject is human.

40. The composition of claim 39, wherein the subject is pregnant.

41. The composition of claim 31, wherein the particle-based control release device comprises a bio-microbur therapeutic delivery platform comprising: a spherical, hollow core having a surface, and a plurality of nanoneedles secured to the surface of the core and extending outwardly therefrom.

42. The composition of claim 41, wherein the core and nanoneedles comprise manganese oxide (MnO₂) or titanium oxide (TiO₂).

43. The composition of claim 41, wherein the core is loaded inside with SSRI encapsulated mesoporous silica nanoparticles.

44. The composition of claim 41, wherein the nanoneedles have an average length of about 1 nm to 100 nm.

45. The composition of claim 41, wherein the bio-microbur has a size in the range of 1 μm to 5 μm.

46. A method of treating or ameliorating the effect of a disorder in a subject, comprising administering to the subject an effective amount of the composition according to claim 31.
